(19) United States
(12) Patent Application Publication
(10) Pub. No.: US 2021/0033472 A1
(43) Pub. Date: Feb. 4, 2021

(54) WEARABLE TAG READER FOR TEMPERATURE-CONTROLLED ENVIRONMENTS

(71) Applicant: BLUECHIIP LIMITED, Scoresby (AU)

(72) Inventors: Scott TURNER, Scoresby (AU); Andrew MCLELLAN, Scoresby (AU); Ian JOHNSTON, Northamptonshire (GB); Henry BLAIN, Dingley Village (AU)

(21) Appl. No.: 16/966,628

(22) PCT Filed: Jan. 30, 2019
(86) PCT No.: PCT/AU2019/050066

§ 371 Date: Jul. 31, 2020

(30) Foreign Application Priority Data
Feb. 1, 2018 (AU) 2018900314

(51) Int. Cl. G01K 1/02; G01K 13/00; G01K 1/14; G01S 19/01; G08C 17/02; G06F 1/16; G06K 7/10

(52) U.S. Cl. CPC G01K 1/024; G01K 13/002; G01K 1/14; G01S 19/01; A61B 90/98; G06F 1/163; G06F 1/1632; G06K 7/10366; G08C 17/02

(57) ABSTRACT

A wearable device is provided for wirelessly reading data in a temperature-controlled environment, the wearable device being adapted to be worn on an upper limb of a user, wherein the wearable device includes an antenna and circuitry adapted to activate the antenna and wirelessly read data from a machine-readable tag in the temperature-controlled environment.

### CROSS REFERENCE TO RELATED APPLICATION

This application claims priority from Australian Provisional Patent Application No. 2018900314 filed on 1 Feb. 2018, the contents of which are to be taken as incorporated herein by this reference.

### TECHNICAL FIELD

This invention relates to wearable devices for wirelessly reading data from machine-readable tags in a temperature-controlled environment and to systems and methods employing these wearable devices. The machine-readable tags may be associated with temperature-sensitive items that require identification and/or location within the temperature-controlled environment. This invention relates particularly but not exclusively to wearable devices that enable wireless reading of data from machine-readable tags in low-temperature or cryogenic environments.

### BACKGROUND OF INVENTION

Certain biological samples are required to be maintained at very low temperatures for long-term storage, such as red blood cells, plasma, bacterial or viral strains, embryos, gametes and extracted DNA to name a few. These samples are typically required to be maintained at temperatures of less than −60° C. to −200° C. To achieve this, the samples are usually placed in vials, cassettes, boxes or other similar vessels and stored within mechanical freezers or in dry ice at temperatures of −60° C. to −150° C., or within cryogenic tanks containing liquid nitrogen at temperatures of less than −150° C. Storage at these temperatures ensures sample integrity, thereby maximising the likelihood of cell viability when thawed.

Similarly, other temperature-sensitive products such as fresh produce, food products, perishables, pharmaceuticals, drugs and chemical compounds must be stored at low temperatures. For example, a typical standard for frozen food products is that they must be stored and handled at temperatures of less than −18° C. The failure to maintain the products at the required temperature may cause their quality to degrade, rendering them inedible or unusable and result in loss of items having significant monetary value and/or importance.

For each of the industries consuming or processing the aforementioned items, it is important to track the items and determine that the items are still presently stored at the correct temperature for quality control at various stages in the supply chain, including storage, processing and transportation. Tracking of the stored items often occurs by removing the item from storage and reading handwritten or printed labels or barcodes. A number of difficulties arise with this approach, including poor writing surfaces, little room for extensive information, difficulty in locating a particular sample amongst the many thousands of items maintained in a storage unit and so on. Furthermore, frost formation on items in cold storage is another problem. Frost formation often occurs due to ingress of air of high humidity through access doors and hatches of a storage unit which mixes with cold air in the storage unit. A build-up of frost can cause degradation of paper labels or cardboard containers commonly used in industry, together with impaired reading of label or barcodes by humans or optical scanners.

Some industries employ the use of machine-readable tags such as Radio Frequency Identification (RFID) tags to track products in their distribution system. RFID tags may be wirelessly read when the RFID tag is brought into proximity with an RFID reader. Wirelessly reading RFID tags may allow information about the tagged product to be quickly obtained and accurately processed. In some industries, hand-held RFID readers are employed for tracking products in a distribution system in ambient environments. Since both of the user's hands are typically required to hold and operate hand-held RFID readers, the user is unable to hold the product or a container storing the product while reading the RFID tag. To address this, RFID readers have been incorporated into wearable garments or clothing to enable the product tag to be more easily read by the user in ambient environments. However, such wearable or hand-held RFID readers are not suitable for use nor can they operate in temperature-controlled environments, such as low-temperature or cryogenic storage facilities.

It would therefore be desirable to provide a wearable device that enables wirelessly reading of machine-readable tags, such as RFID tags, in temperature-controlled environments, particularly in low-temperature or cryogenic storage facilities, and which ameliorates and/or overcomes one or more problems and/or inconveniences of the prior art.

A reference herein to a patent document or any other matter identified as prior art, is not to be taken as an admission that the document or other matter was known or that the information it contains was part of the common general knowledge as at the priority date of any of the claims.

### SUMMARY OF INVENTION

According to one aspect of the present invention, there is provided a wearable device for wirelessly reading data in a temperature-controlled environment, the wearable device being adapted to be worn on an upper limb of a user, wherein the wearable device includes an antenna and circuitry adapted to activate the antenna and wirelessly read data from a machine-readable tag in the temperature-controlled environment.

Preferably, the wearable device is further adapted for use in temperature-controlled environments having temperatures in the range of −200° C. to −0° C. This desirably enables the wearable device to be used in low-temperature or cryogenic storage environments.

In some embodiments, the wearable device further includes a housing for the antenna and the circuitry, wherein the housing is shaped to be worn on the upper limb of the user. The wearable device may be adapted to be retrofit to a glove. Alternatively, the wearable device may be a glove.

The housing may include an antenna housing portion for housing the antenna. The antenna housing portion may be shaped to be worn on a hand of the user. Preferably, the antenna housing portion is shaped to be worn on a thumb or finger, or more preferably, a thumb tip or finger tip of the user. The housing may also include a circuitry housing portion for housing the circuitry. Preferably, the circuitry housing portion is shaped to be worn on a hand, a wrist or an arm of the user.

The housing may include a protective layer for isolating one or both of the antenna and the circuitry from the temperature-controlled environment to minimise one or both of thermal shock and fluid damage in the temperature-controlled environment. The housing may also include one or more conduits for directing fluid away from one or both of the antenna and the circuitry for drainage externally of the wearable device to minimise one or both of thermal shock and fluid damage in the temperature-controlled environment. The housing may also include a hydrophobic coating to deter fluid collection on a surface thereof for visibility of the wearable device in the temperature-controlled environment.

In some embodiments, the circuitry is adapted to wirelessly read the data by detecting changes in resonance of the machine-readable tag. The machine-readable tag may include a plurality of resonant members encoding an identifier. The circuitry may be adapted to applying an excitation signal to the machine-readable tag through the antenna that vibrates the resonant members to wirelessly read the identifier. The circuitry may be adapted to automatically activate the antenna by detection of a magnetic field associated with the machine-readable tag. Preferably, the machine-readable tag includes a MEMS (micro-electromechanical systems) structure.

The wearable device may be further adapted for providing directed illumination towards the machine-readable tag or the wearable device for visibility in the temperature-controlled environment. The wearable device may include one or more illuminators for providing the directed illumination. Preferably, the one or more illuminators are positioned near the antenna for directed illumination during reading of the machine-readable tag.

The antenna may be extendable or removable from the device for positioning near the machine-readable tag in the temperature-controlled environment. The wearable device may further include a second antenna that is extendable or removable from the wearable device for positioning near the machine-readable tag in the temperature-controlled environment. The second antenna may be housed in a wand shaped to be held by the user.

The wearable device may further include a docking station having one or more tools operable by the user for positioning a container associated with the machine-readable tag near the antenna.

The wearable device may further include a vial reader for receiving a vial associated with the machine-readable tag. The vial reader may include an antenna operable to be activated by circuitry for wirelessly reading data from the machine-readable tag.

In some embodiments, the wearable device further includes one or more temperature sensors for measuring a temperature of a container associated with the machine-readable tag. The wearable device may include a processing device adapted to one or more of: receive temperature measurements from the one or more temperature sensors; monitor viability of a temperature-sensitive item stored in the container; and provide feedback to the user on the viability of the temperature-sensitive item.

The wearable device may further include a processing device in communication with the circuitry. The processing device may be adapted to provide feedback to the user on the data wirelessly read by the circuitry. The feedback may include one or both of whether the data received is sufficient for reading of the machine-readable tag and an identifier of the machine-readable tag.

The wearable device may further include a processing device in communication with a remote computing device. The processing device may be adapted to receive location information of the machine-readable tag from the remote computing device and to output the location information to the user for locating the machine-readable tag in the temperature-controlled environment. The wearable device may further include a location device for providing location information of the wearable device. The processing device may be further adapted to receive the location information of the wearable device and to output directions to the user for locating the machine-readable tag in the temperature-controlled environment.

In some embodiments, the wearable device further includes an identification device for providing an identification of the wearable device. The identification may be readable by a remote computing device for verifying the identification of the wearable device using a database and authorising user access to the temperature-controlled environment based on the verification.

According to another aspect of the present invention, there is provided a system for wirelessly reading data in a temperature-controlled environment, the system including: a wearable device for wirelessly reading data in a temperature-controlled environment, the wearable device being adapted to be worn on an upper limb of a user, wherein the wearable device includes an antenna and circuitry adapted to activate the antenna and wirelessly read data from a machine-readable tag in the temperature-controlled environment; and a remote computing device in communication with the wearable device, wherein the remote computing device is adapted to receive data wirelessly read from the machine-readable tag in the temperature-controlled environment.

In some embodiments, the wearable device further includes one or more temperature sensors for measuring a temperature of a container associated with the machine-readable tag and the remote computing device is further adapted to one or more of: receive temperature measurements from the one or more temperature sensors; monitor viability of a temperature-sensitive item stored in the container; and transmit feedback on the viability of the temperature-sensitive item to the wearable device.

The remote computing device may be further adapted to transmit location information of the machine-readable tag to the wearable device for locating the machine-readable tag in the temperature-controlled environment. The wearable device may further include a location device for providing location information of the wearable device. The remote computing device may be further adapted to receive the location information of the wearable device and to track the location of the wearable device in the temperature-controlled environment.

The wearable device may further include an identification device for providing an identification of the wearable device, and the remote computing system may be further adapted to read the identification of the wearable device, verify the identification of the wearable device using a database and authorise user access to the temperature-controlled environment.

The remote computing device may be further adapted to update inventory records using data received from the wearable device.

According to another aspect of the present invention, there is provided a method for wirelessly reading data in a temperature-controlled environment, the method including the steps of: providing a wearable device for wirelessly reading data in a temperature-controlled environment, the wearable device being adapted to be worn on an upper limb of a user, wherein the wearable device includes an antenna and circuitry adapted to activate the antenna and wirelessly read data from a machine-readable tag in the temperature-controlled environment; and wirelessly reading data from the machine-readable tag using the circuitry in the temperature-controlled environment.

In some embodiments, wirelessly reading data includes the step of detecting changes in resonance of the machine-readable tag using the circuitry. The machine-readable tag may include a plurality of resonant members encoding an identifier, and detecting changes in resonance may include the steps of: applying an excitation signal to the machine-readable tag through the antenna using the circuitry that vibrates the resonant members; and wirelessly reading the identifier using the circuitry. The method may further include the steps of: detecting a magnetic field associated with the machine-readable tag using the circuitry; and automatically activating the antenna based on the detected magnetic field.

The method may further include the step of providing directed illumination towards the machine-readable tag or the wearable device for visibility in the temperature-controlled environment.

In some embodiments, the method further includes the steps of: extending or removing the antenna from the wearable device; and positioning the antenna near the machine-readable tag in the temperature-controlled environment. The wearable device may further include a second antenna, and the method may further include the steps of; extending or removing the second antenna from the wearable device; and positioning the second antenna near the machine-readable tag in the temperature-controlled environment.

The wearable device may further include a docking station having one or more tools, and the method further includes the step of: positioning a container associated with the machine-readable tag near the antenna using the one or more tools.

The wearable device may further include a vial reader having an antenna operable to be activated by circuitry, and the method may further include the steps of: receiving a vial associated with the machine-readable tag in the vial reader; and wirelessly reading data from the machine-readable tag using the circuitry in the temperature-controlled environment.

In some embodiments, the wearable device further includes one or more temperature sensors for measuring a temperature of a container associated with the machine-readable tag, and the method further includes the step of: measuring the temperature of the container using the one or more temperature sensors.

In some embodiments, the wearable device further includes a processing device, and the method further includes one or more of the following steps: receiving, using the processing device, temperature measurements from the one or more temperature sensors; monitoring, using the processing device, viability of a temperature-sensitive item stored in the container; and providing, using the processing device, feedback to the user on the viability of the temperature-sensitive item.

The method may further include the steps of: receiving, using a remote computing device in communication with the wearable device, temperature measurements from the one or more temperature sensors; monitoring, using the remote computing device, viability of the temperature-sensitive item stored in the container; and transmitting, using the remote computing device, feedback on the viability of the temperature-sensitive item to the wearable device for the user.

The wearable device may further include a processing device in communication with the circuitry, and the method may further include the step of: providing, using the processing device, feedback on the data wirelessly read by the circuitry. The feedback may include one or both of whether the data received is sufficient for reading of the machine-readable tag and an identifier of the machine-readable tag.

The wearable device may further include a processing device in communication with a remote computing device, and the method may further include the steps of: receiving, using the processing device, location information of the machine-readable tag from the remote computing device; and outputting, using the processing device, the location information to the user for locating the machine-readable tag in the temperature-controlled environment.

In some embodiments, the wearable device further includes a location device for providing location information of the wearable device, and the method further includes the steps of: receiving, using the processing device, location information of the wearable device from the location device; and outputting, using the processing device, directions to the user for locating the machine-readable tag in the temperature-controlled environment.

The wearable device may further include a location device for providing location information of the wearable device, and the method may further include the steps of: receiving, using a remote computing device in communication with the wearable device, location information of the wearable device from the location device; and tracking, using the remote computing device, the location of the wearable device in the temperature-controlled environment.

In some embodiments, the wearable device further includes an identification device for providing an identification of the wearable device, and the method further includes the steps of: reading, using a remote computing device in communication with the wearable device, the identification of the wearable device; verifying, using the remote computing device, the identification of the wearable device using a database; and authorising, using the remote computing device, user access to the temperature-controlled environment based on the verification.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the claims appended hereto.

FIG. 1 is a schematic diagram of a wearable device for wirelessly reading data in a temperature-controlled environment according to an embodiment of the invention, showing an antenna and circuitry.

FIG. 2 is a perspective view of the wearable device of FIG. 1 showing a wrist band and fingertip antenna according to another embodiment of the invention.

FIG. 3 is a perspective view of the wearable device of FIG. 2 retrofit to a glove.

FIG. 4 is a perspective view of the wearable device of FIG. 1 being hand-mounted with a fingertip antenna according to another embodiment of the invention, showing the wearable device retrofit to a glove.

FIG. 5A is a plan view of the wearable device of FIG. 1 from a topside and showing a housing covering the wrist and thumb and a thumb tip antenna, and

FIG. 5B is a plan view from an underside of the wearable device of FIG. 5A retrofitted to a glove, according to another embodiment of the invention.

FIG. 6A is a plan view of the wearable device of FIG. 1 from a topside and showing a housing covering the wrist, two fingers and thumb and a thumb tip antenna, and FIG. 6B is a plan view from an underside of the wearable device of FIG. 6A retrofitted to a glove, according to another embodiment of the invention.

FIG. 7 is a perspective view of the wearable device of FIG. 1 being a glove and showing a display device, according to another embodiment of the invention.

FIG. 8 is a cross-sectional view of the wearable device of FIG. 7 along the line 8-8′ through the glove in the x-direction showing a protective layer and conduits for fluid drainage according to another embodiment of the invention.

FIG. 9 is a perspective view of the wearable device of FIG. 7 showing a thumb tip antenna according to another embodiment of the invention.

FIG. 10 is a perspective view of the wearable device of FIG. 7 showing a tag illuminator and a device illuminator according to another embodiment of the invention.

FIG. 11A is a perspective view showing reading of a machine-readable tag attached to a handle of a tank using the wearable device of FIG. 7, and FIG. 11 B is an enlarged view of the tank handle, according to another embodiment of the invention.

FIG. 12A is a perspective view showing reading of a machine-readable tag attached to a handle of a tower using the wearable device of FIG. 7, and FIG. 12B is an enlarged view of the tower handle, according to another embodiment of the invention.

FIG. 13A is a perspective view showing reading of a machine-readable tag of a container stored in a tower using the wearable device of FIG. 7, and FIG. 13B is an enlarged view of the container, according to another embodiment of the invention.

FIG. 14A is a perspective view showing reading of a machine-readable tag of a container using a wand having an antenna of the wearable device of FIG. 7, and FIG. 14B is a perspective view of the wearable device of FIG. 14B showing the wand stored in a docking station, according to another embodiment of the invention.

FIG. 15 is a perspective view showing reading of a machine-readable tag of a vial using the wearable device of FIG. 7 according to another embodiment of the invention.

FIG. 16 is a perspective view showing reading of a machine-readable tag of a container using the wearable device of FIG. 7 with visual, auditory and sensory feedback, according to another embodiment of the invention.

FIG. 17A is a perspective view of the wearable device of FIG. 1 being a glove and having a thumb antenna, and FIG. 17B is a perspective view showing reading of a machine-readable tag of a container using the wearable device of FIG. 17A with an indicator light for feedback, according to another embodiment of the invention.

FIGS. 18A-C are plan views of the wearable device of FIG. 7 from a topside showing reading of a machine-readable tag of a container with visual feedback on a display device, according to embodiments of the invention.

FIGS. 19A-B are plan views of a display device of the wearable device of FIG. 7 having display screens oriented horizontally (FIG. 19A) and vertically (FIG. 19B) according to embodiments of the invention, and showing an identifier of the machine-readable tag.

FIGS. 20A-E are perspective views of the wearable device of FIG. 1 from a topside according to embodiments of the invention, illustrating varying arrangements of the circuitry component housing and a display device, with FIG. 20A also showing a docking station for one or more tools and FIG. 20E omitting a display device.

FIG. 21 is a perspective view of the wearable device of FIG. 7 showing a vial reader and a display device according to another embodiment of the invention.

FIG. 22 is a side view of the wearable device of FIG. 1 showing a fingertip antenna, a vial reader, a display device and an optical device according to another embodiment of the invention, being retrofitted to a glove.

FIG. 23 is a schematic diagram of the elements and circuitry of the wearable device of FIGS. 1 to 22 and machine-readable tag shown in FIGS. 11 to 18, according to another embodiment of the invention.

FIG. 24 is a schematic diagram of an antenna coupled to a chip of a machine-readable tag of FIG. 23.

FIG. 25 is an isometric view of an embodiment of a resonant member forming part of the machine-readable tag of FIG. 24.

FIG. 26 is a graphical representation of the frequency response of the machine-readable tag shown in FIGS. 24 and 25.

FIG. 27 is a schematic diagram of the elements and circuitry of the wearable device depicted in FIGS. 1 to 23, according to another embodiment of the invention.

FIG. 28 is a detailed schematic diagram of the elements and circuitry of the wearable device depicted in FIGS. 1 to 23, according to another embodiment of the invention.

FIG. 29 is a schematic diagram of a system for wirelessly reading data in a temperature-controlled environment according to an embodiment of the invention, showing a machine-readable tag, a wearable device and a remote computing device.

FIG. 30 is a schematic diagram of the system of FIG. 29 according to an embodiment of the invention, showing the wearable device in communication with a server or workstation through a network device.

FIG. 31 shows a flow chart illustrating steps in a method for wirelessly reading data in a temperature-controlled environment according to an embodiment of the invention.

### DETAILED DESCRIPTION

Embodiments of the invention are discussed herein by reference to the drawings which are not to scale and are intended merely to assist with explanation of the invention.

The inventive device, system and method may be useful in enabling wireless reading of machine-readable tags, such as RFID tags, in a temperature-controlled environment, such as a low-temperature or cryogenic storage facility. The temperature-controlled environment may include temperatures from −200° C. to 0° C. The machine-readable tags may be associated with containers for storing temperature-sensitive items. The temperature-sensitive items may include biological samples, such as red blood cells, plasma, bacterial or viral strains, gametes and embryos, produce, such as fresh produce, food products and perishables, pharmaceuticals, drugs and chemical compounds, and other temperature-sensitive items that require, cold, ultra-cold (i.e. less than −80° C.) or cryogenic storage. The inventive device, system and method may thus be used in conjunction with identification and tracking of containers storing temperature-sensitive items throughout a distribution system.

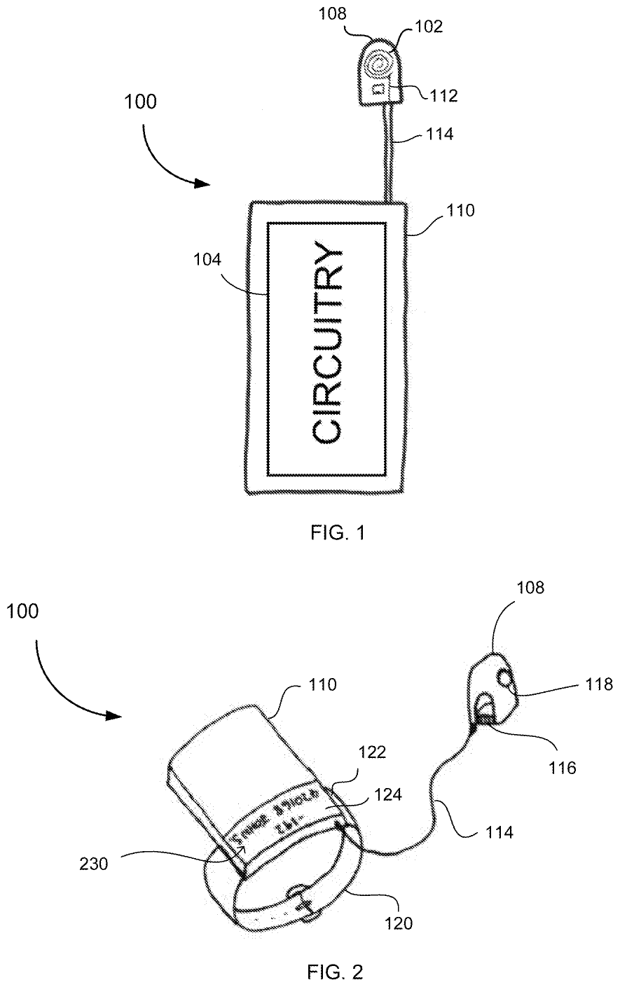

FIG. 1 is a schematic diagram of a wearable device 100 for wirelessly reading data in a temperature-controlled environment according to a preferred embodiment of the invention. The wearable device 100 is adapted to be worn on an upper limb 410 of a user 400. The wearable device 100 includes an antenna 102 and circuitry 104 adapted to activate the antenna 102 and wirelessly read data from a machine-readable tag 200 in the temperature-controlled environment.

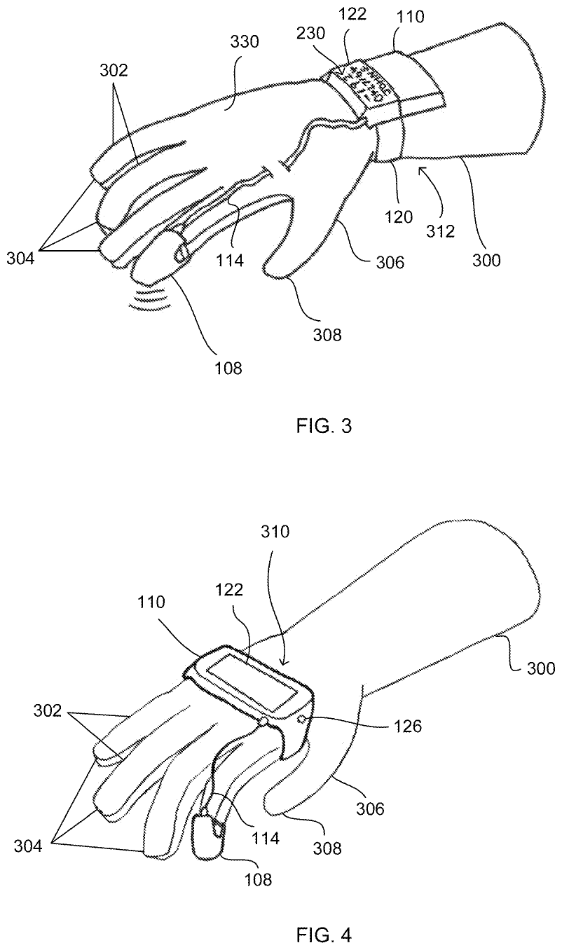

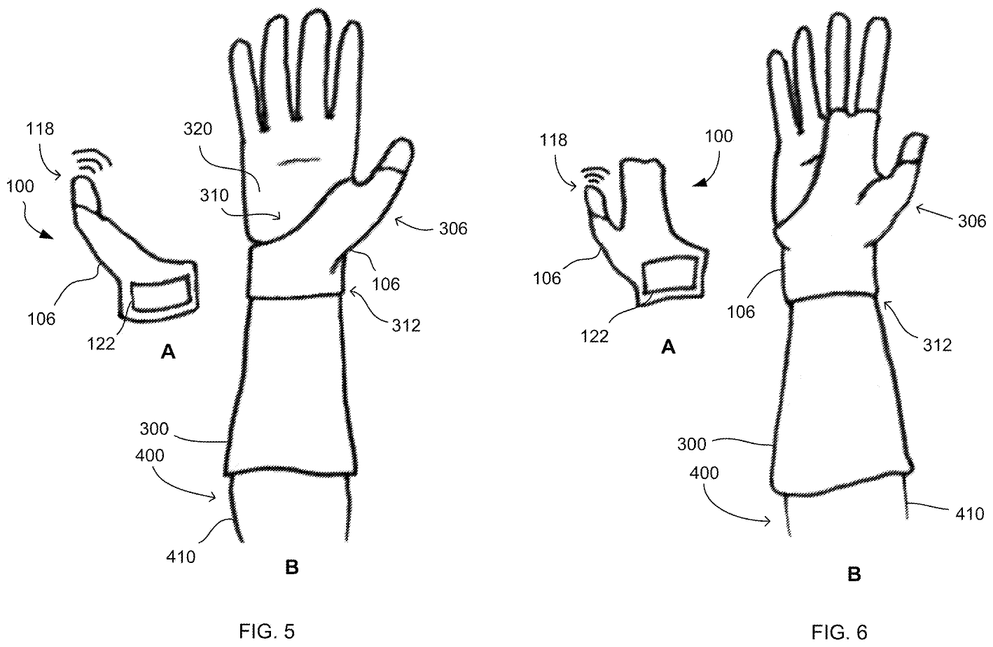

FIGS. 2 and 3 are perspective views of an embodiment of the wearable device 100 having a wrist band 120 and a fingertip antenna housing 108. As shown in FIG. 3, the wearable device 100 may be worn over a glove 300, and may preferably be retrofit to the glove 300. Ideally, the glove 300 worn by the user 400 is suitable to withstand extreme temperatures of heat or cold in temperature-controlled environments to protect the upper limb 410 of the user 400 (see also FIGS. 5 and 6). The glove 300 may be a laboratory or cryogenic glove suitable to withstand temperatures from −200° C. to 0° C. Although not shown, the wearable device 100 may be directly worn on the upper limb 410 of the user 400 without a glove 300 for use in ambient environments. The wearable device 100 may be adapted to be worn on the left or right hand of the user 400, over a left or right-handed glove 300. Preferably, the wearable device 100 is worn over the glove 300 or retrofit to the glove 300 in such a way that enables use as personal protective equipment (PPE). Furthermore, the wearable device 100 may desirably include flexible connections so as to provide free movement and dexterity in temperature-controlled environments, such as where the use of PPE is required.

The wearable device 100 may include a housing 106 for the antenna 102 and the circuitry 104, which is shaped to be worn on the upper limb 410 of the user 400 (see also FIGS. 5 and 6). FIGS. 1 to 3 illustrate that the housing 106 may include an antenna housing portion 108 for housing the antenna 102 that is shaped to be worn on a hand of the user 400. Preferably, the antenna housing portion 108 is shaped to be worn on a finger of the user 400 over a finger 302 of the glove 300. The antenna housing portion 108 may be shaped to only extend over a fingertip 304 of the glove 300 as shown in FIG. 3. The antenna housing portion 108 may be fixed over the fingertip 304 by a retainer 116, which may include a resilient material. In other embodiments, the antenna housing portion 108 may be shaped to extend along the entire finger 302, be positioned on different fingers than the index finger or be shaped to be worn around a thumb or thumb tip of the user 400, such as over the thumb 306 or thumb tip 308 of the glove 300 of FIGS. 2 and 3.

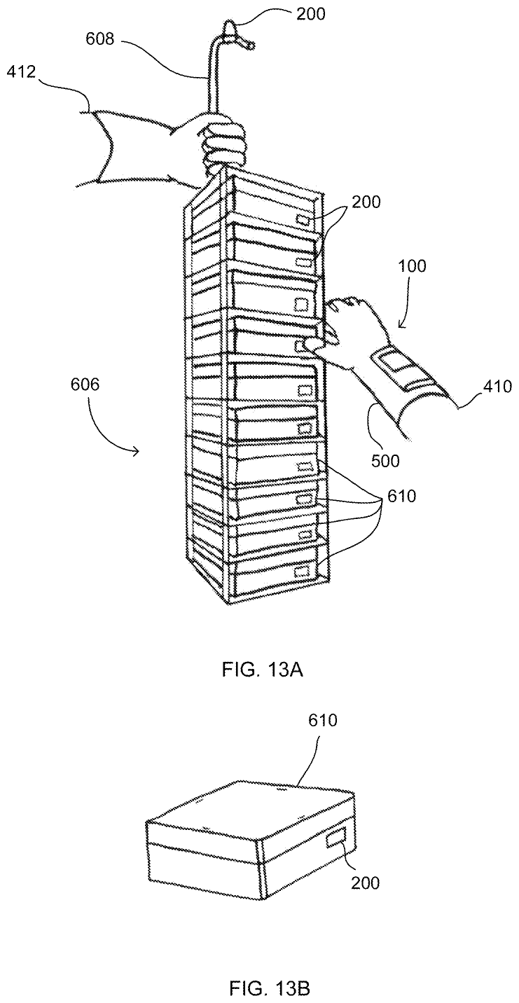

The antenna housing portion 108 may also include a reader head 118 for housing the antenna 102 as shown in FIG. 2. The reader head 118 may form a primary reader of the wearable device 100 as shown in the detailed schematic diagram of FIG. 30. Although not visible in FIG. 3, the reader head 118 may be positioned on an underside 320 of the glove 300 at the fingertip 304 as indicated by the antenna signal lines (see also FIG. 5B). Alignment of the reader head 118 with the glove fingertip 304 enables intuitive control of the wearable device 100 as the user 400 can point their finger or reach their hand towards the machine-readable tag 200 to make the reading. This also advantageously allows the user 400 to read the machine-readable tag 200 with the wearable device 100 on one arm 410 while providing a free arm 412 for the user 400 to perform another task, such as holding a container 600 (see also FIG. 13A).

In some embodiments, the wearable device 100 includes a circuitry housing portion 110 for housing the circuitry 104, which is shaped to be worn on a hand, a wrist or an arm of the user 400. Although the circuitry 104 is housed separately from the antenna 102 in these embodiments, a person skilled in the art would readily appreciate that the circuitry 104 may be housed together with the antenna 102, such as in the antenna housing portion 108. As shown in FIGS. 2 and 3, the circuitry housing portion 110 may include a wrist band 120 for attaching around the user's wrist by a buckle and clip arrangement. Alternatively, the wrist band 120 may attach by Velcro straps, a hook and loop arrangement or resilient material or other means as would be appreciated by a person skilled in the art. As shown in FIG. 3, the wrist band 120 is attached around a wrist portion 312 of the glove 300 although the buckle and clip arrangement is not visible from the topside 330 of the glove 300. The circuitry housing portion 110 may be shaped to include smooth surfaces on the wrist band 120 and a display device 122. The smooth surfaces may minimise physical damage to the wearable device 100 by reducing the likelihood of catching or interference with external objects in the temperature-controlled environment.

FIGS. 1 to 3 also show that the wearable device 100 may include a connector housing 114 for housing a connector 112 for communication between the antenna 102 and circuitry 104. The connector housing 114 may be connected to the antenna housing portion 108 and the circuitry housing portion 110. Additionally, the connector housing 114 may be affixed to the glove 300, such as by threading through a loop on the glove 300 to reduce movement during use as shown in FIG. 3. The connector housing 114 is preferably maintained with some flexibility to enable the user 400 to clench and outstretch their hand with the glove 300 on without tensioning the connector 112.

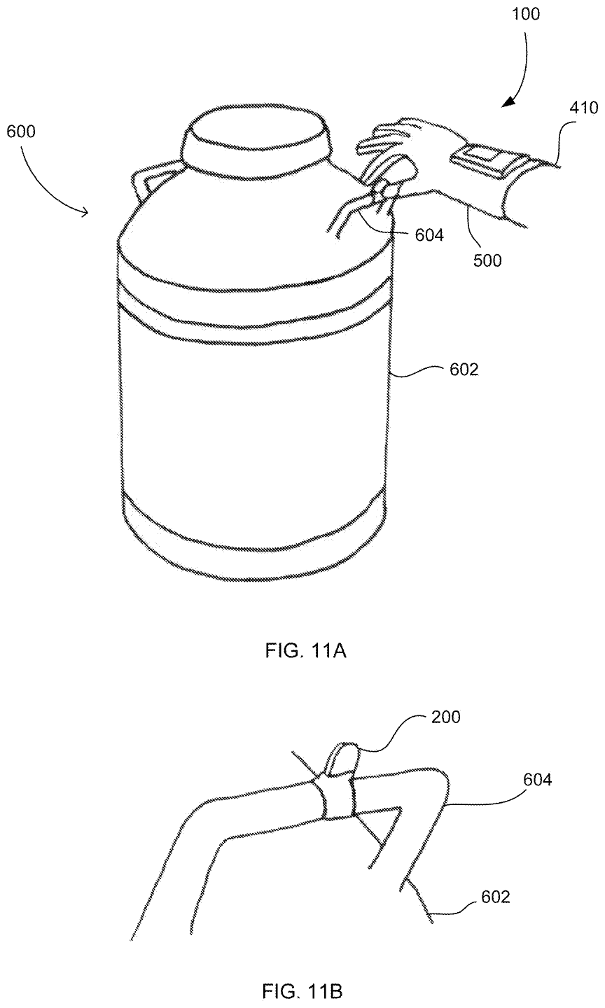

In addition to the circuitry 104, the circuitry housing portion 110 may also house a display device 122 having a display screen 124 as shown in FIGS. 2 and 3. The display screen 124 may be visible on the topside 330 of the glove 300 as shown in FIG. 3 for ease of viewing by the user 400. The display device 122 may be adapted to display feedback to the user 400 on the data wirelessly read by the circuitry from the machine-readable tag 200. The feedback may include whether the data read is sufficient for reading of the machine-readable tag 200 and an identifier 230 of the machine-readable tag 200. The identifier 230 may provide information regarding a container 600 associated with the machine-readable tag 200 or a temperature-sensitive item stored in the container 600 (see also FIG. 11). For example, the identifier 230 may include a temperature, a number and a name, as shown on the display screen 124 in FIGS. 2 and 3. Preferably, the identifier 230 may provide information regarding a temperature-sensitive item, such as a biological sample, stored in the container 600. Although a display device 122 is included in these embodiments, the wearable device 100 may omit a display device 122 and/or feedback means.

FIG. 4 illustrates another embodiment of the wearable device 100 in which the circuitry housing portion 110 is shaped to be worn around the hand of the user 400 over the glove 300. The circuitry housing portion 110 is particularly shaped to be worn around a palm portion 310 of the glove 300. Additionally, the circuitry housing portion 110 may include a display device 122 in which the display screen 124 occupies a large proportion of the circuitry housing portion 110 on a topside 330 of the glove 300. This larger display screen 124 may be useful for visibility to the user 400 in the temperature-controlled environment. The circuitry housing portion 110 may also include a depressible button 126 for switching the wearable device 100 on or off as shown in FIG. 4.

FIGS. 5 and 6 illustrate embodiments of the wearable device 100 having a single housing 106 for the antenna 102 and the circuitry 104. FIG. 5A illustrates a housing 106 of the wearable device 100 adapted to be worn over the wrist and thumb of the user 400 and having a thumb tip reader head 118, viewed from a topside 330 of a glove 300. FIG. 5B illustrates the wearable device 100 of FIG. 5A retrofit to a glove 300 over a wrist portion 312, part of the palm portion 310 and the thumb 306 of the glove 300, and viewed from an underside 320 of the glove 300. FIGS. 6A-B illustrate a similar embodiment although the housing 106 is also adapted to be worn additionally over part of the palm portion 310 that extends to the index and second fingers 302 of the glove 300.

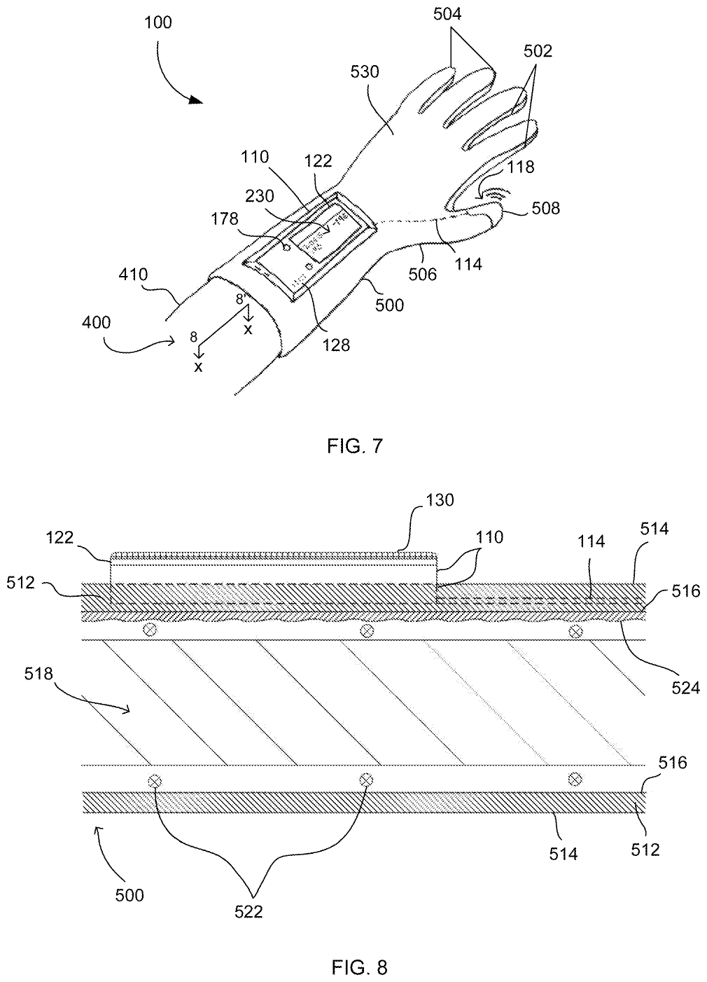

FIGS. 7 to 21 illustrate embodiments of the invention where the wearable device 100 is a glove 500 instead of being worn over, attached or retrofit to a glove 300 as shown in FIGS. 2 to 6. It should be appreciated that the features described herein with respect to the embodiments of FIGS. 7 to 21 may also be incorporated into the wearable device 100 of FIGS. 2 to 6, as would be understood by a person skilled in the art. Preferably, the components of the wearable device 100 are integrally formed with the glove 500. The glove 500 may be shaped to be worn over a hand and part of the arm 410 of the user 400 as shown in FIG. 7. Similar to the glove 300, the glove 500 is preferably suitable to withstand extreme temperatures of heat or cold in temperature-controlled environments to protect the upper limb 410 of the user 400. For example, the glove 500 may be constructed with materials and joints to withstand temperatures from −200° C. to 0° C. for use in low-temperature or cryogenic environments.

The wearable device 100 with glove 500 may be adapted to be worn on the left or right hand of the user 400, over a left or right-handed glove 500. Preferably, the glove 500 is sized to provide a close fit over the user's hand for use as personal protective equipment (PPE). Furthermore, the wearable device 100 with glove 500 may desirably include flexible connections so as to provide free movement and dexterity in temperature-controlled environments, such as where the use of PPE is required. The glove 500 may also include large components, such as buttons, display device 122 and tools 136, for user interaction with the glove 500 while the user 400 is wearing PPE.

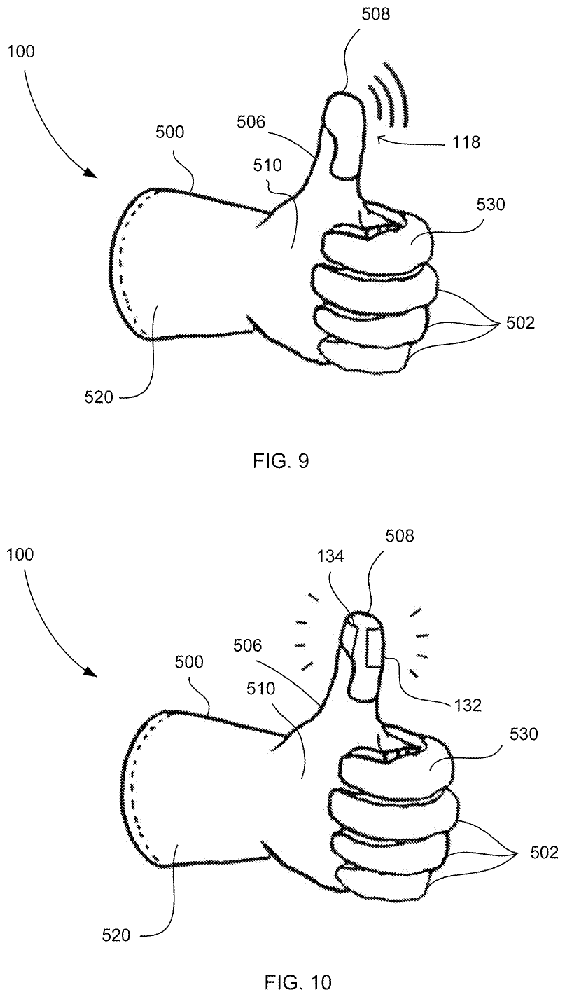

As shown in FIG. 7, the antenna 102 of the wearable device 100 may be housed in a reader head 118 positioned within a thumb tip 508 of a thumb 506 of the glove 500. The reader head 118 is preferably positioned on an underside 520 of the glove 500 as indicated by the antenna signal lines (see also FIGS. 9 and 10). The connector housing 114 for housing the connector 112, which connects the antenna 102 and the circuitry 104, may be located internally of the glove 500 as indicated by the dotted lines in FIGS. 7 and 8. The connector housing 114 may be located internally of the glove 500 in order to minimise physical damage to the wearable device 100, such as by interference or catching of the flexible housing 114 on external objects in the temperature-controlled environment. Additionally, the circuitry housing portion 110 remains partially exposed on a topside 530 of the glove 500 so that the user 400 may access the display device 122 and/or various input/output devices as will be described in more detail below (see also FIGS. 9 and 10).

FIG. 8 illustrates a cross-sectional view of the wearable device 100 of FIG. 7 along the line 8-8′ through the glove 500 in the direction denoted by the ‘x’ references. As shown in FIG. 8, the glove 500 may include a housing 512 for housing the antenna, circuitry and/or connector housing portions 108, 110 and 114 of the wearable device 100. The circuitry housing portion 110 may partially protrude from an outer surface 514 of the glove housing 512 in order to be accessible by the user 400, such as for visibility of the display device 122 with a display screen 124.

In some embodiments, the housing 106, 512 of the wearable device 100 may be adapted for minimising one or both of thermal shock and fluid damage to the wearable device 100 in the temperature-controlled environment. The wearable device 100 may be exposed to extreme temperatures of heat or cold, which may result in thermal shock to the electrical components. For example, a user 400 wearing device 100 may enter or exit the temperature-controlled environment from an ambient environment causing a rapid increase or decrease in temperature of the wearable device 100 which may damage the electrical components. Furthermore, due to the extreme temperatures in the temperature-controlled environment, the wearable device 100 may be exposed to fluids that may damage the electrical components, such as vapour or condensate at high temperatures or melting ice and liquids at low temperatures.

In order to combat the extreme temperatures, the housing 106 of the wearable device 100 of the embodiments shown in FIGS. 2 to 6 may include a protective layer for isolating one or both of the antenna 102 and circuitry 104 from the temperature-controlled environment (not shown). The protective layer may surround one or both of the antenna 102 and the circuitry 104 so as to provide insulation from extreme temperatures and/or separation from fluids in the temperature-controlled environment to minimise one or both of thermal shock and fluid damage to the wearable device 100. The protective layer may include waterproof and/or heatproof material for example. The housing 106 of the wearable device 100 as shown in FIGS. 2 to 6 may also include one or more conduits for directing fluid away from one or both of the antenna 102 and the circuitry 104 for drainage externally of the wearable device 100 (not shown). In the event that fluid, such as vapour, condensate, melted ice or liquids, enters the housing 106 of the wearable device 100, the conduits may be shaped and arranged so as to direct the fluid away from the electronic components to minimise one or both of thermal shock and fluid damage to the wearable device 100 in the temperature-controlled environment.

Referring now to FIG. 8, the glove housing 512 of the wearable device 100 of the embodiments of FIGS. 7 to 21 may include a protective layer 524 located between inner surfaces 516 of the glove housing 512. The protective layer 524 may enable the components of the wearable device 100 housed in the glove housing 512 to remain isolated from the temperature-controlled environment. The protective layer 524 may provide a barrier to thermal shock and fluid damage to the wearable device 100. For example, fluids may enter the glove 500 between the inner surfaces 516 of the glove housing 512 prior, after or during wear of the glove 500 by the user 400. The protective layer 524 will further insulate the components of the wearable device 100 from thermal shock due to fluids entering or exiting the glove 500 and from fluid damage due to fluid entering the glove 500. The protective layer 524 may include waterproof and/or heatproof material for example.

Advantageously, the glove 500 may also provide one or more conduits 522 for directing fluid away from one or both of the antenna 102 and circuitry 104 for drainage externally of the wearable device 100. Preferably, a plurality of conduits 522 are provided as shown in FIG. 8. The conduits 522 are ideally positioned between the inner surfaces 516 of the glove housing 512. The conduits 522 may be adapted to enable drainage of fluid that enters the glove 500 prior, after or during wear of the glove 500 by the user 400 as described above. The conduits 522 may include channels that direct fluid to the outer surface 514 of the glove housing 512 for drainage (not shown). The drainage system in the glove 500 provided by the conduits 522 is preferably separated and isolated from the components of the wearable device 100 for minimising one or both of thermal shock and fluid damage to the wearable device 100.

The housing 106 or 512 may include a hydrophobic coating to deter fluid collection on a surface thereof for visibility in the temperature-controlled environment. Fluid collection may cause frosting due to freezing of liquids in low-temperature environments or misting due to condensation in high-temperature environments. This may result in the display device 122 being partially or fully obscured from the user's view. A hydrophobic coating 130 may be included on the display device 122 as shown in FIG. 8 to advantageously enables the display device 122 to remain visible to the user 400 in the temperature-controlled environment. The hydrophobic coating 130 may include various substances to deter fluid collection thereon, particularly water collection, as would be appreciated by a person skilled in the art.

In some embodiments, the glove 500 may also include a removable insert 518 for receiving at least part of the upper limb 410 of the user 400 and maintaining the upper limb 410 separate from the housing 512 of the glove, including the conduits 522 and protective layer 524, as shown in FIG. 8. The removable insert 518 may provide additional protection for the components of the wearable device 100 housed in the glove housing 512. The removable insert 518 may be waterproof and include non-porous material for preventing fluid entering the housing 512 of the glove 500. Ideally, the removable insert 518 extends through the entire length of the glove 500 including to the fingers 502 and thumb 506 (not shown). The removable insert 518 may be washable and reusable. In some embodiments, the insert 518 is permanent and non-removable from the glove 500.

Furthermore, the wearable device 100 may include a housing 106, 512 adapted for minimising physical damage to the components of the wearable device 100 in the temperature-controlled environment. For example, the wearable device 100 may include smooth surfaces to reduce interference with external objects in the temperature-controlled environment. This may minimise the likelihood of catching of parts of the wearable device 100 and tearing or damage to those parts in the temperature-controlled environment. For example, in the embodiments of FIGS. 2 to 8, the housing 106, 512 includes smooth surfaces to reduce interference or catching on external objects.

The wearable device 100 may also be constructed to resist external forces in the temperature-controlled environment. The wearable device 100 with housing 106, 512 may be made of materials that are resistive of forces that may damage the housing portions 108, 110 and 114 and/or the electrical components that are housed therein. A person skilled in the art will appreciate the various materials that are suitable for providing robustness to the housing 106. The housing 106, 512 may be reinforced with protective structures that prevent direct contact of the electrical components against the housing 106, 512 in order to minimise physical damage to the wearable device 100 (not shown).

FIG. 9 is a perspective view of the wearable device 100 with glove 500 as illustrated in FIGS. 7 and 8, illustrating a reader head 118 housing the antenna 102 positioned near a thumb tip 508 on the underside 520 of the glove 500. The reader head 118 is not visible since it is positioned beneath the outer surface 514 of the glove housing 512. In some embodiments, the reader head 118 may either protrude from the outer surface 514 of the glove 500 or form a dimple on the outer surface 514 of the glove 500 for visibility by the user 400, similar to the embodiment shown in FIG. 2. Additionally/alternatively, the outer surface 514 of the glove 500 may include a marker or symbol for indicating the position of the reader head 118. This may be desirable for enabling intuitive reading of the machine-readable tag 200 by the user 400. In some embodiments, the antenna 102 and reader head 118 may alternatively be positioned in the thumb 506, fingers 502, fingertips 504 or palm portion 510 of the glove 500.

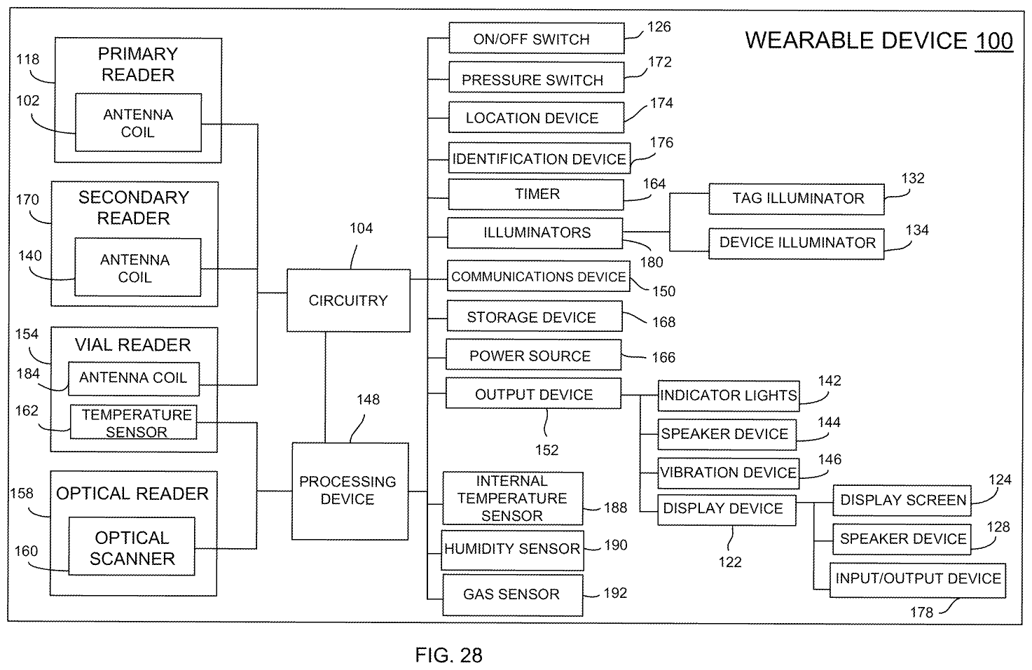

FIG. 10 shows another embodiment of the wearable device 100 with glove 500 of FIGS. 7 to 9. The wearable device 100 may be further adapted for providing directed illumination towards the machine-readable tag 200 or the wearable device 100 for visibility in the temperature-controlled environment. This may be desirable as temperature-controlled environments often have reduced or poor visibility due to frost, fog and/or condensate, or are dark or poorly lit. The wearable device 100 may include one or more illuminators 180 for providing the directed illumination (see also FIG. 28). Preferably, the illuminators 180 are positioned near the antenna 102 or reader head 118 so that the machine-readable tag 200 and wearable device 100 may be illuminated during reading of the machine-readable tag 200.

As shown in FIG. 10, the wearable device 100 may include a tag illuminator 132 for providing directed illumination towards the machine-readable tag 200 and a device illuminator 134 for providing directed illumination towards the wearable device 100. The device illuminator 134 may be positioned near the antenna 102 although on the topside 530 of the glove 500, and optionally, opposite to the tag illuminator 132 on the underside 520 of the glove 500. The tag and device illuminators 132, 134 may include LED lights or any kind of lights as would be appreciated by a person skilled in the art. Although the tag and device illuminators 132, 134 are shown positioned on the thumb tip 508, they could be located on any part of the glove 500.

FIGS. 11 to 15 illustrate examples of the wearable device 100 with glove 500 of FIGS. 7 to 10 in use by the user 400 in the temperature-controlled environment. The machine-readable tag 200 may be associated with a container 600 for storing a temperature-sensitive item in the temperature-controlled environment. The machine-readable tag 200 may be attached, affixed or formed integral with the container 600. FIGS. 11A-B illustrate that the container 600 may be a tank 602, such as a cryogenic or low-temperature tank, having a machine-readable tag 200 attached to a handle 604 of the tank 602. The user 400 may contact the thumb tip 508 of the glove 500 having the antenna 102 with the machine-readable tag 200 on the handle 604 of the tank 602 to wirelessly read the machine-readable tag 200 as shown in FIG. 11A.

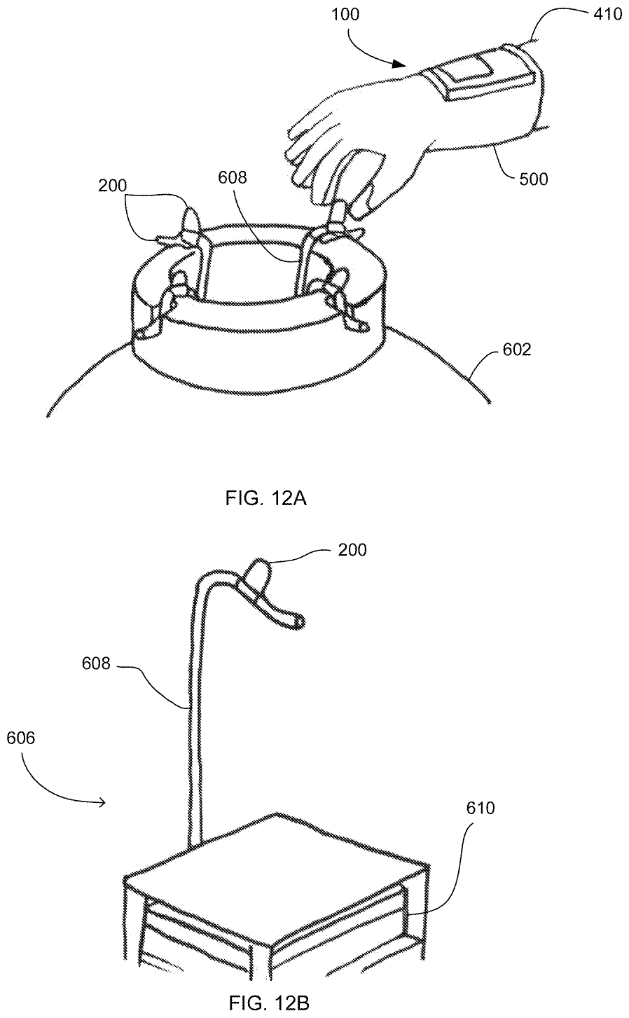

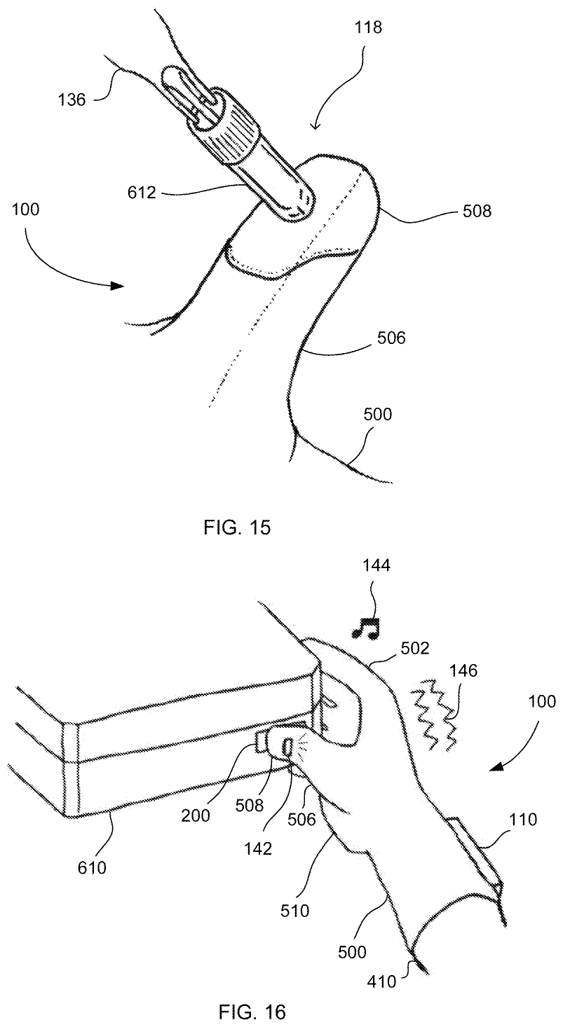

In another example, FIGS. 12A-B illustrate that the container 600 may include a tower 606 for storing a plurality of boxes 610. The boxes 610 may store one or more vials 612 and each vial 612 may store a temperature-sensitive item (see also FIG. 15). A number of towers 606 may be stored in a tank 602 as illustrated in FIG. 12A. Each tower 606 may include one or more machine-readable tags 200. The user 400 may contact the thumb tip 508 of the glove 500 having the antenna 102 with the machine-readable tag 200 attached to a handle 608 of one of the towers 606 to wirelessly read the machine-readable tag 200 as shown in FIG. 12A.

In other embodiments, the user 400 may use a free arm 412 to grasp a handle 608 of a tower 606 and remove the tower 606 from the tank 602 as shown in FIG. 13A. Preferably, the free arm 412 is the user's dominant arm for use in lifting and moving towers 606 and their non-dominant arm is used for wearing the wearable device 100 with glove 500. This beneficially enables the user 400 to more quickly and efficiently perform their task of reading of the machine-readable tags 200 in the temperature-controlled environment. It also minimises the time that the temperature-sensitive items stored in the containers 600 are exposed to increased or decreased temperatures, such as from tanks 602 or towers 606, thus maximising the viability of the items. The user 400 may be wearing the wearable device 100 with glove 500 on their other arm 410. The user 400 may contact the thumb tip 508 of the glove 500 having the antenna 102 with one or more machine-readable tags 200 associated with boxes 610 stored in the tower 606 to wirelessly read the machine-readable tags 200 as shown in FIGS. 13A.

In some embodiments, the wearable device 100 is further adapted for positioning of the antenna 102 near the machine-readable tag 200 to facilitate access to the machine-readable tag 200 in the temperature-controlled environment. The antenna 102 may be extendable or removable from the wearable device 100 for positioning near the machine-readable tag 200 (not shown). The antenna 102 may be housed in a reader head 118 as shown in FIG. 2, which is extendable by wire from the antenna housing portion 108. Additionally/alternatively, the reader head 118 and/or antenna housing portion 108 may be extendible from the circuitry housing portion 110 by means of flexibility in the connector 112 and/or connector housing 114.

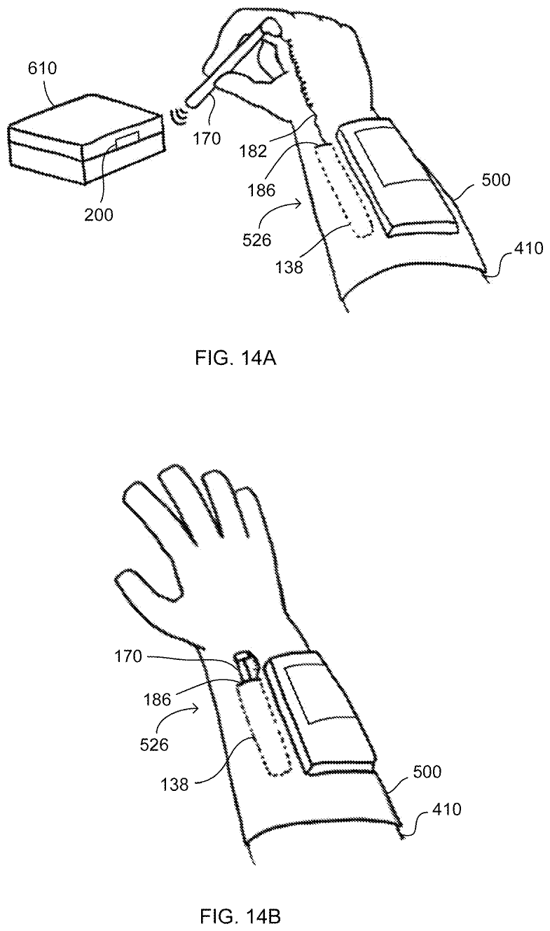

FIGS. 14A-B show another embodiment of the wearable device 100 that includes a secondary reader 170 having a second antenna that is extendible from the wearable device 100 by a cable 182 for positioning near the machine-readable tag 200 of a box 610 (see also FIG. 28). The secondary reader 170 may be stored in a docking station 138 and be extended from the docketing station 138 for use by the user 400 as a wand. The second antenna 140 may be activated by the circuitry 104 to wirelessly read data from the machine-readable tag 200. The secondary reader 170 may omit the cable 182 in some embodiments and wirelessly transmit data from the machine-readable tag to the glove 500. The antenna 102 and/or second antenna 140 may desirably be extendable or removable in order for the user 400 to access machine-readable tags 200 in small spaces and/or in environments of danger to the user 400. For example, the user 400 may use the antenna 102 and/or second antenna 140 to access machine-readable tags 200 stored in tanks 602 filled with liquid nitrogen without endangering the user 400.

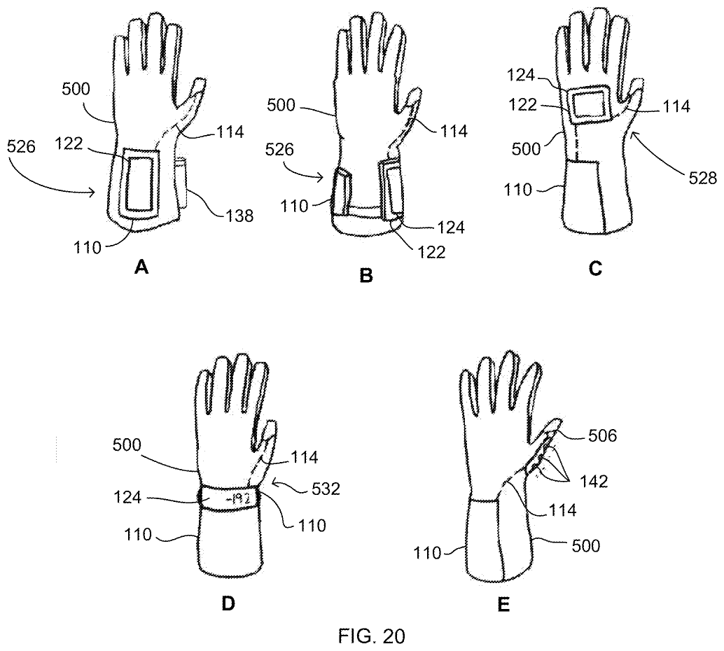

As shown in FIGS. 14A-B and 20A, the wearable device 100 may include a docking station 138 for storing one or more tools 136, such as the secondary reader or wand 170. The docking station 138 may be positioned within the housing 512 of the glove 500 and include an opening 186 on the outer surface 514 of the glove to provide access to the tools 136. The docking station 138 may be advantageously embedded in the glove housing 512 in order to provide smooth surfaces and reduce the likelihood of interference with external objects in the temperature-controlled environment. In other embodiments, the docking station 138 may include a sleeve for receiving the one or more tools 136 and retaining them near the glove 500 as shown in FIG. 20A. The sleeve may include an opening having elastic material for releasably storing the tools 136 on the glove 500. Alternatively, the docking station 138 may include a mounted portion on the glove 500 with one or more retainers, preferably being clips, for releasably storing the tools 136. The docking station 138 is preferably positioned on an arm portion 526 of the glove 500 to one side of the display device 122 as shown in FIGS. 14A-B and 20A. In other embodiments, the docking station 138 may be positioned anywhere on the arm portion 526 so as to avoid interference with the antenna 102.

In some embodiments, the wearable device 100 is further adapted for positioning of the machine-readable tag 200 near the antenna 102 to facilitate access to the tag 200 in the temperature-controlled environment. The wearable device 100 may include one or more tools 136 operable by the user 400 for positioning a container 600 associated with the tag 200, such as a vial 612, near the antenna 102. FIG. 15 illustrates another embodiment where the container 600 is a vial 612 for storing a temperature-sensitive item. The vial 612 may include a machine-readable tag 200 near a base thereof, which is attached or formed integrally with the vial 612 (not shown). The user 400 may use a tool 136 for holding the vial 612 near or in contact with the thumb tip 508 of the glove 500 having the antenna 102 for reading the machine-readable tag 200 of the vial 612. The user 400 may hold the tool 136 using their free arm 412 while the glove 400 is worn on their other arm 410. The tool 136 may include tweezers, tongs or specialised grasping tools for holding the vial as shown in FIG. 15.

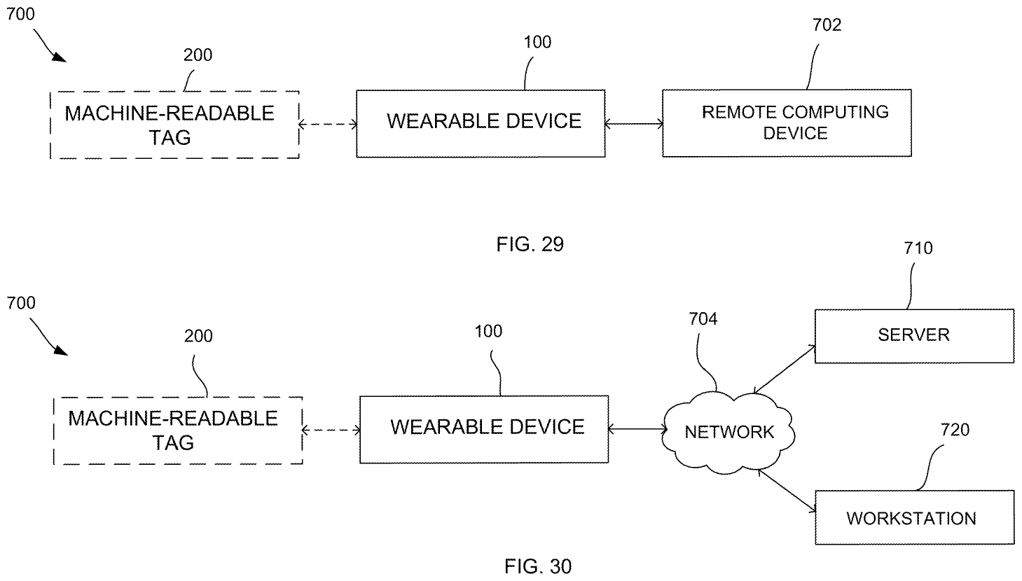

In some embodiments, the wearable device 100 is further adapted to provide feedback on the data wirelessly read by the circuitry 104. The wearable device 100 may include a processing device 148 in communication with the circuitry 104 for providing the feedback (see also FIGS. 29 and 30). The processing device 148 may be adapted to provide feedback to the user 400 including one or both of whether the data received is sufficient for reading of the machine-readable tag 200 and an identifier 230 of the machine-readable tag 200. The wearable device 100 may also include an output device 152 for providing one or more of visual, auditory or sensory feedback to the user 400. The processing device 148 and electronic components of the wearable device 100 will be described in more detail below.

FIG. 16 shows an example of the user 400 wearing the wearable device 100 with glove 500 and wirelessly reading a machine-readable tag 200 on a box 610. The output device 152 may include one or more indicator lights 142 for providing visual feedback to the user 400. An indicator light 142 may be positioned on a thumb tip 508 of a thumb 506 of the glove 500 as shown in FIG. 16. Preferably, the indicator light 142 is positioned in the reader head 118. Although the indicator light 142 is positioned at this location, it could be positioned anywhere on the glove 500 so as to be visible to the user 400. The antenna 102 may also be housed in the thumb tip 508 and directed by a reader head 118 towards the box 610 on an underside 520 of the glove 500 (see also FIGS. 9 and 10). Preferably, the indicator light 142 is positioned on the topside 530 of the glove 500 for visibility to the user 400 when reading the machine-readable tag 200. As such, the one or more indicator lights 142 may be positioned to direct light towards the user 400 for viewing in the temperature-controlled environment. The indicator light 142 may include an LED light or any kind of suitable light as would be appreciated by a person skilled in the art. If the data received is not sufficient for reading of the machine-readable tag 200, the indicator light 142 may flash. Otherwise, the indicator light 142 may illuminate for a predetermined time, such as 5 seconds, to indicate to the user 400 that the data received is sufficient for reading of the machine-readable tag 200.

The output device 152 may also include a speaker device 144 for providing auditory feedback or a vibration device 146 for providing sensory feedback to the user 400. The speaker device 144 and vibration device 146 may be housed in the housing 512 of the wearable device 100. Preferably, the speaker device 144 and vibration device 146 are positioned in the reader head 118. Although the speaker device 144 is shown near the fingers 502 of the glove 500 and the vibration device 146 is shown near the back of the palm 510 of the glove 500, they could be positioned anywhere on the glove 500. The speaker device 144 may provide a tone, music or language to provide the feedback to the user 400. For example, if the data received is not sufficient for reading of the machine-readable tag 200, the speaker device 144 may provide a short tone or spoken language such as “POOR READING”. Otherwise, the speaker device 144 may provide a longer tone or spoken language such as “GOOD READING”. The vibration device 146 may provide vibrational or tactile feedback to the user 400 that can be felt by the user 400 through the glove 500. The vibration device 146 may vibrate for a predetermined time, such as 5 seconds, if the data received is sufficient for reading of the machine-readable tag 200, and otherwise provide no vibration.

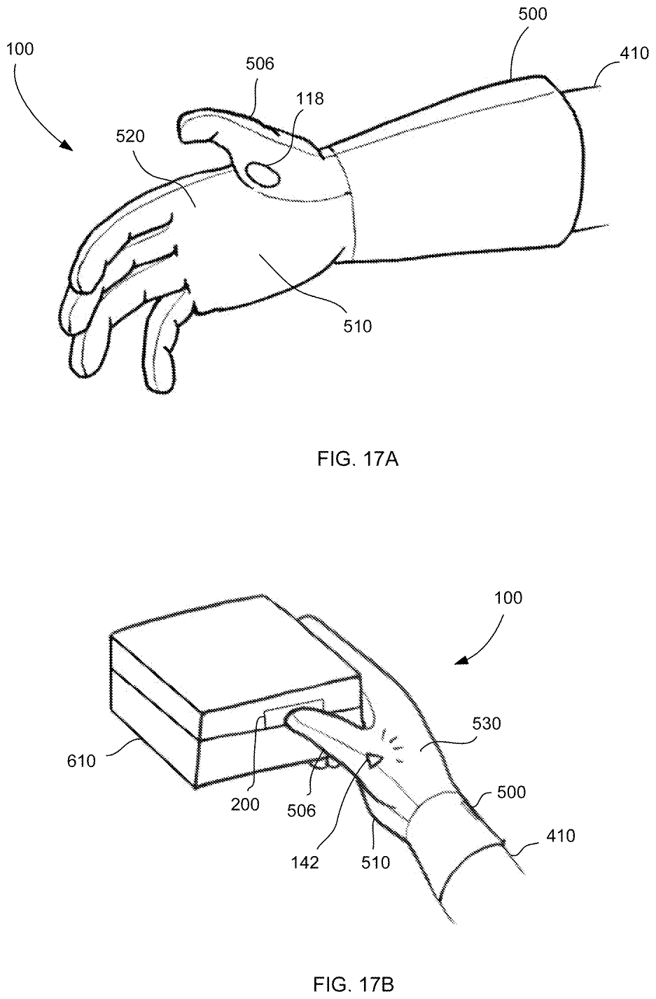

FIGS. 17A-B illustrate another embodiment of the wearable device 100 having the glove 500 for reading a machine-readable tag 200 on a box 610. In contrast to the previous embodiments, the wearable device 100 includes a reader head 118 housing the antenna 102 positioned on the thumb 506 at an underside 520 of the glove 500, as shown in FIG. 17A. When the user 400 holds the box 610 with their hand in the glove 500 as shown in FIG. 17B, the machine-readable tag 200 may be read due to the proximity of the reader head 118 to the machine-readable tag 200. It is not necessary for the reader head 118 with antenna 102 to contact the machine-readable tag 200. Instead, the close proximity of the antenna 102 may enable the reading of the machine-readable tag 200. Additionally, the indicator light 146 may be located on the thumb 506 instead of the thumb tip 508 of the glove 500 as shown in FIG. 17B. Alternatively, the indicator light 146 may be positioned at any part of the glove 500 for visibility by the user 400.

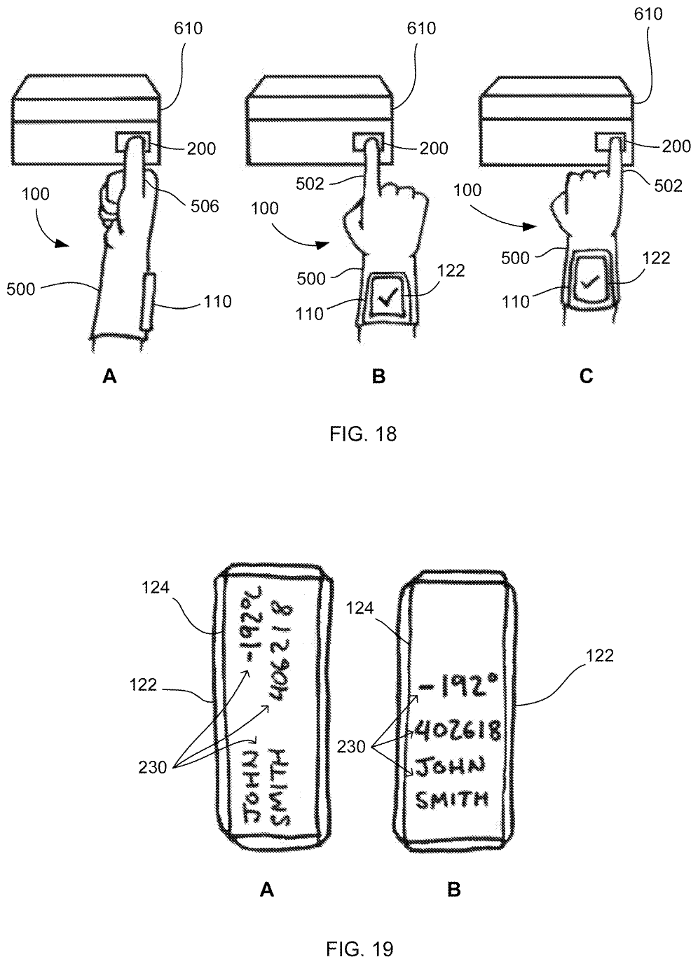

In some embodiments, the output device 152 may be a display device 122 as shown in FIGS. 2 and 3. The display device 122 may provide one or more of visual, auditory or sensory feedback to the user 400. The display device 122 may be housed in the circuitry housing portion 110 of the wearable device 100 and include a display screen 124 for providing visual feedback by the user 400. FIGS. 18A-C illustrate examples of the user 400 wearing the wearable device 100 having the glove 500 for reading of a machine-readable tag 200 on a box 610, and the display of visual feedback to the user 400 on the display device 122. In these embodiments, the antenna 102 is positioned in one or more of the fingers 502 of the glove 500. FIG. 18A shows an incorrect method for reading the machine-readable tag 200 using a thumb 506 of the glove 500. Although not visible, the display device 122 may display on the display screen 124 a cross ‘X’ to the user 400 to indicate that the data received is not sufficient for reading of the machine-readable tag 200. FIGS. 18B and 18C indicate correct methods for reading the machine-readable tag 200 by using one of the fingers 502 of the glove 500 having the antenna 102. When the data read is sufficient for reading of the machine-readable tag 200, the display device 122 may display on the display screen 124 a tick mark to the user 400 as shown in FIGS. 18B and 18C.

If the processing device 148 determines that the data read is sufficient for reading of the machine-readable tag 200, the processing device 148 may read an identifier 230 of the machine-readable tag 200. The tag 200 may be adapted to provide a machine-readable identifier 230, as shown in FIGS. 2 and 3, so as to provide identification information of a container 600 associated with the tag 200. The identifier 230 may include information for a temperature-sensitive item stored in the container 600. For example, the information may include the item number, type, preparation date, expiry date and location information. FIGS. 19A-B illustrate display devices 122 with display screens 124 for displaying the identifier 230 read by the processing device 148. FIG. 19A illustrates a horizontal display screen 124 whereas FIG. 19B illustrates a vertical display screen 124. The identifier 230 displayed may include a temperature and an identification number and a name for the container 600. The information from the identifier 230 may be associated with a temperature-sensitive item, such as a biological sample, stored in the temperature-controlled environment. Advantageously, the temperature-sensitive item may be identified without the need to remove it from the temperature-controlled environment, thereby reducing the likelihood of comprising viability of the item.

FIGS. 20A-E illustrate variations on the embodiments of the wearable device 100 with glove 500 as shown in FIGS. 7 to 19, illustrating a topside 530 of the gloves 500. FIG. 22A shows the wearable device 100 with glove 500 with a docking station 138 for storing one or more tools 136 for holding a container 600 near the antenna 102.

FIGS. 20B and 20C illustrate variations on the location of the display device 122 in the glove 500. FIG. 20B shows that the display device 122 may be housed separately from the circuitry housing portion 110. The display device 122 may be housed on right side of the glove 500 and the circuitry housing portion 110 may be housed on the left side of the glove 500, or vice versa, on an arm portion 526 of the glove 500. FIG. 20C shows an embodiment of the wearable device 100 with glove 500 in which the display device 122 is housed separately in a hand portion 528 of the glove 500.

FIG. 20D illustrates an embodiment of the wearable device 100 with glove 500 showing that the circuitry housing portion 110 may include the display device 122 with a display screen 124 on a wrist portion 532 of the glove 500. The display screen 124 may occupy the majority of the space on the circuitry housing portion 110 on the topside 530 of the glove 500. The display screen 124 may display a temperature for the indicator 230 of the machine-readable tag 200 as shown in FIG. 20D. The display screen 124 may be gyroscopically mounted for viewing at different angles by the user 400, which may be particularly useful in temperature-controlled environments where certain angles of the wearable device 100 may be obscured from the user's view. Accordingly, the wearable device 100 advantageously enables improved visibility of the wearable device 100 and/or feedback shown on the display screen 124 in the temperature-controlled environment.

FIG. 20E illustrates an embodiment of the wearable device 100 with glove 500 that excludes a display device 122. Instead, the wearable device 100 is adapted to provide feedback on the data read to the user 400 by a plurality of indicator lights 142 along the thumb 506 of the glove 500. Additionally/alternatively, the wearable device 100 may provide auditory or sensory feedback to the user 400. Furthermore, in some embodiments, no feedback on the data read may be provided to the user 400.

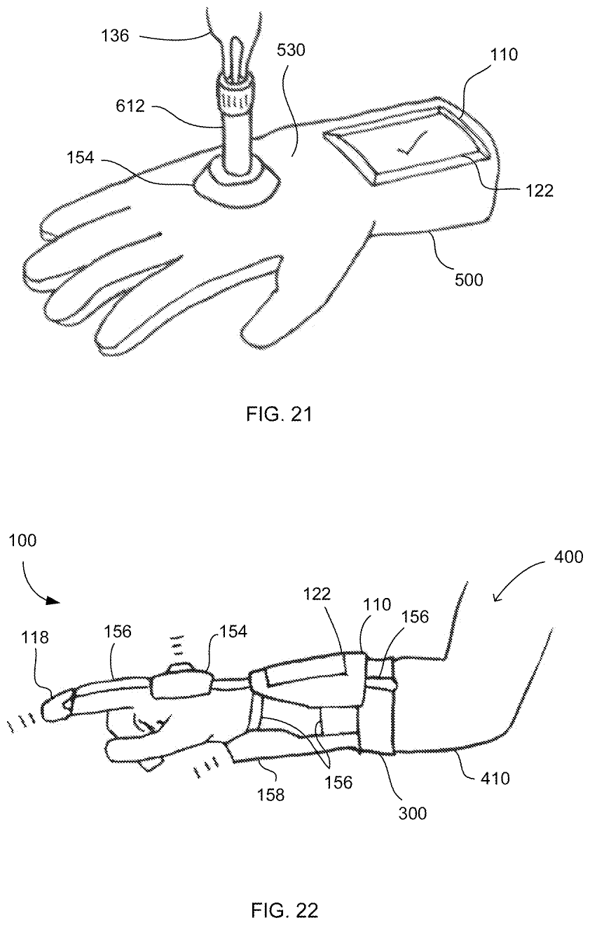

In some embodiments, the wearable device 100 may include a vial reader 154 for receiving a vial 612 associated with the machine-readable tag 200, as shown in FIG. 21. The vial reader 154 may include an antenna 184 operable to be activated by circuitry 104 for wirelessly reading data from the machine-readable tag 200 (see also FIG. 28). The user 400 may operate a tool 136 for holding the vial 612 and positioning it near the antenna 184 for reading of the machine-readable tag 200. The vial reader 154 may be shaped for receiving the vial 612, such as by having a rounded lip for receiving the end of the vial 612. Preferably, the vial reader 154 is positioned on a topside 530 of the glove 500. As shown in FIG. 21, feedback on the data read by the vial reader 154 may be provided on the display device 122, which shows a tick mark to indicate that the data read is sufficient to read the machine-readable tag 200.

In some embodiments, the vial reader 154 may be provided instead of having a primary reader 118 positioned in one of the fingers 502, thumb 506 or palm 510 of the glove 500 ora secondary reader 170. Alternatively, the vial reader 154 may be provided in addition to the reader head 118 and/or secondary reader 170 and include at least its own antenna 184 for reading the machine-readable tag 200 for the vial 612. The circuitry 104 may be adapted for activating and wirelessly reading data from the antenna 102 in the reader head 118 and/or the antenna 184 in the vial reader 154. Alternatively, the wearable device 100 may include further circuitry for activating and wirelessly reading data from the antenna 184 in the vial reader 154.

In some embodiments, the wearable device 100 may further include one or more temperature sensors 162 for measuring a temperature of a container 600 associated with the machine-readable tag 200 (see also FIG. 28). The vial reader 154 may include the one or more temperature sensors 162 for measuring a temperature of the vial 612. Additionally/alternatively, the temperature sensor 162 may be included in the antenna housing portion 108 of the wearable device 100. It may be useful for the temperature of the container 600 to be measured and monitored to assess the viability of a temperature-sensitive item stored in the container 600. For example, if the container 600, such as a vial 612, has been removed from a tank 602 or tower 606, the temperature of the temperature-sensitive item may increase or decrease from an ideal storage temperature. Monitoring the exposure of the container 600 to particular temperatures may thus be useful for indicating to the user 400 when, for example, a vial 612 should be returned to the tank 602 or tower 606. The wearable device 100 may also include a timer 164 (see also FIG. 28), provided by software or hardware, which provides feedback to the user 400, such as through the display device 122, regarding the time that the container 600 has been exposed to non-ideal storage temperatures. The timer 164 may also indicate when the vial 612 should be returned to the tank 602 or tower 606 for maintaining viability of the temperature-sensitive item. The temperature sensor 162 may include optical temperature sensors including laser and/or infra-red, wire temperature sensors including thermocouple, thermistor and/or resistance temperature detectors (RTD) or any other sensor type as would be known to a person skilled in the art.

In some embodiments, the wearable device 100 may further include one or more humidity sensors 190 for measuring humidity of the surrounding environment near the container 600 (see also FIG. 28). The humidity sensor 190 may be included in the vial reader 154 and/or the antenna housing portion 108 of the wearable device 100. Alternatively, the humidity sensor 190 may be located anywhere on the wearable device 100. It may be useful for the humidity of the environment surrounding the container 600 to be measured and monitored to assess the moisture content near the container 600 and determine the potential for frost build-up, such as in low-temperature environments, on the container 600 itself or a temperature-sensitive item stored therein. Monitoring humidity may enable feedback to be provided to the user 400 who can instigate countermeasures to the build-up of frost. The humidity sensor 190 may be a capacitive, resistive or thermally conductive sensor or any other sensor type as would be known to a person skilled in the art.

In some embodiments, the wearable device 100 may further include one or more gas sensors 192 for measuring gas concentration of the surrounding environment near the wearable device 100 (see also FIG. 28). Preferably, the gas sensor 192 is positioned on the housing 106 of the wearable device 100 or the glove housing 512 in a location near to the user's head. For example, the gas sensor 192 may be positioned on an arm portion 526 of the glove 500. It may be useful for the gas concentration of the environment surrounding the wearable device 100 to be measured and monitored to assess if the surrounding environment is safe for the user 400. Preferably, the gas sensor 192 is adapted to measure oxygen concentration in the surrounding environment, although may also measure toxic gases such as carbon monoxide and nitrogen dioxide. The gas sensor 192 may be an electrochemical, infrared or thermally conductive sensor or any other sensor type as would be known to a person skilled in the art.

FIG. 22 illustrates an embodiment of the wearable device 100 which is retrofit to an existing glove 300. The wearable device 100 may include the primary reader or reader head 118 having the antenna 102 on a thumb tip, a vial reader 154, a circuitry housing portion 110 having a display device 122 and an optical reader 158. The optical reader 158 may include an optical scanner 160 for wirelessly reading data representative of one or both of indicia and alphanumeric data, such as from a barcode (see also FIG. 28). Each of the components may be electrically and/or physically connected by straps 156 of the wearable device 100. The componentry may be attached over or retrofit to an existing glove 300 by means of the straps 156. Preferably, the straps 156 include resilient material for attaching the wearable device 100 to the glove 300. Alternatively, the wearable device 100 with each of its components may be formed integrally with the glove 500 (not shown).

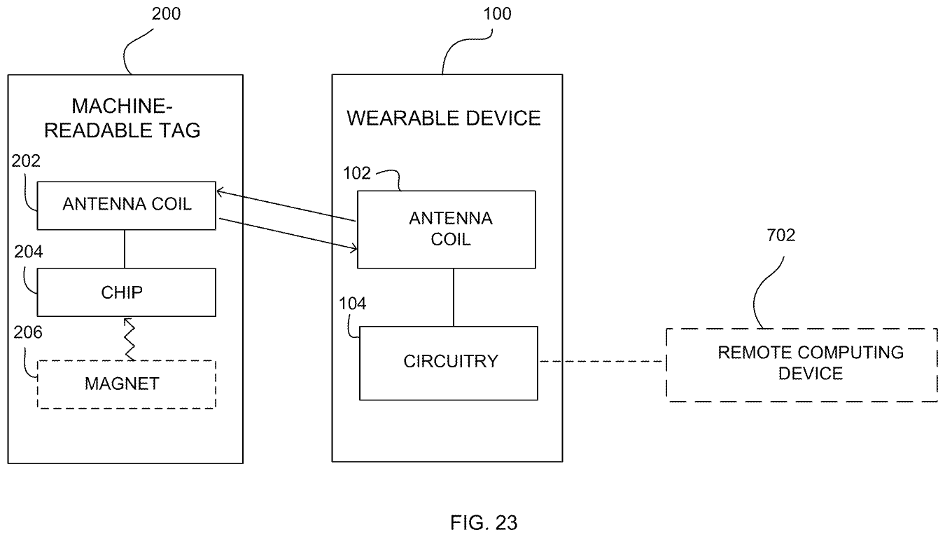

FIG. 23 shows a schematic diagram of the components and circuitry of the wearable device 100 and machine-readable tag 200 of the embodiments as previously described. The machine-readable tag 200 may include an antenna coil 202 in communication with a chip 204. The machine-readable tag 200, for example, may be an RFID tag 200 having an RFID chip 204. The wearable device 100 notably includes an antenna coil 102 and associated circuitry 104. The circuitry 104 of the wearable device 100 may be adapted to wirelessly read data from the machine-readable tag 200 by detecting changes in resonance of the machine-readable tag 200.

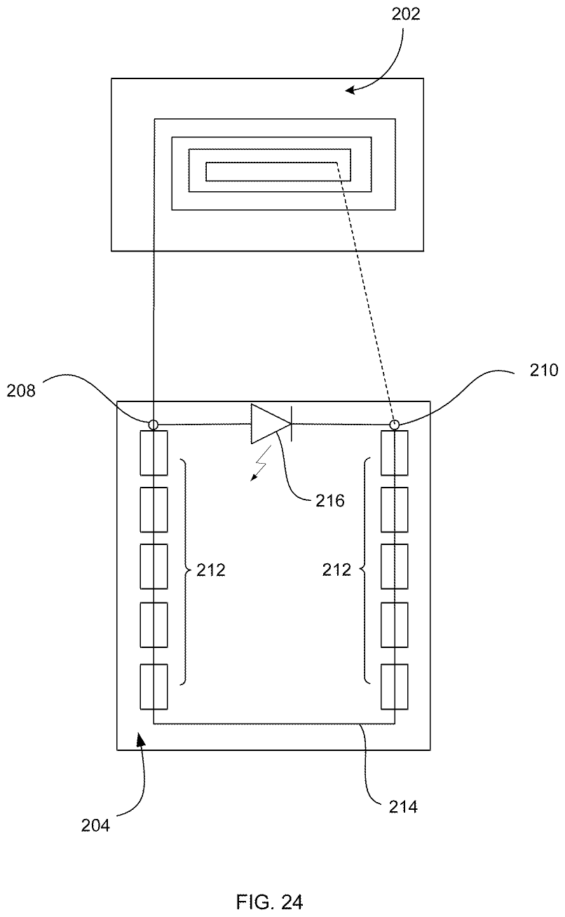

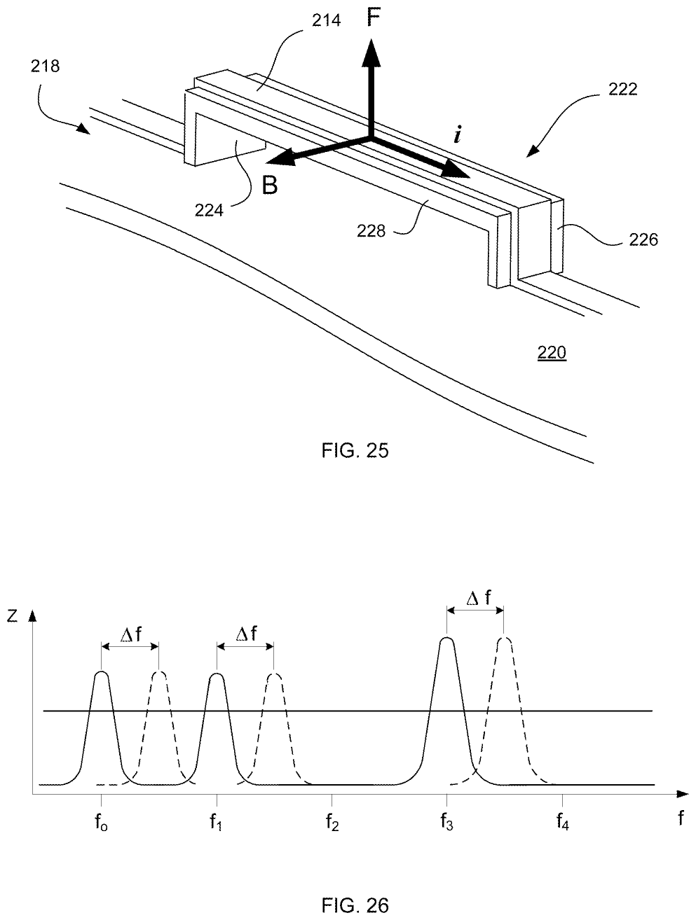

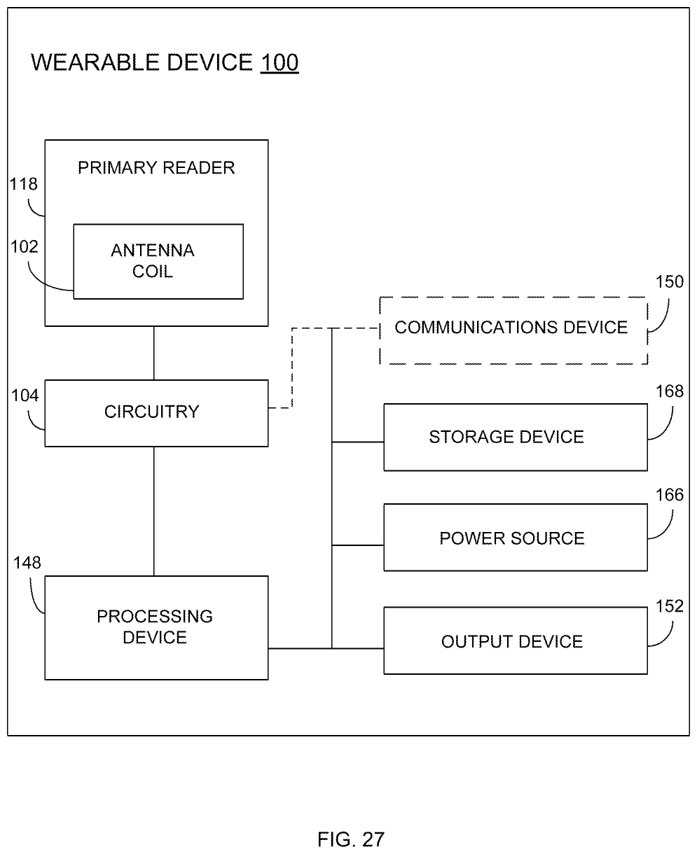

FIG. 24 illustrates the machine-readable tag 200 of FIG. 23 that includes a plurality of resonant members 212 in the chip 204 encoding an identifier 230. The plurality of resonant members 212 may each have a particular resonant frequency and have different resonant frequencies from each other. The resonant members 212 may be micro-mechanical vibratable members 218 as shown in FIG. 25. A common electrical conductor 214 runs along the resonant members 212 and is a section of the chip 204 of the machine-readable tag 200, including three u-shaped sections corresponding to the resonant members 212. The resonant members 212 may be caused to vibrate by an applied excitation signal generated by the circuitry 104 through the antenna 102 that induces an alternating current in the electrical conductor 214 by means of Faraday induction via the antenna coil 202 of the machine-readable tag 200. An LED light 216 may indicate when alternating current is induced in the electrical conductor 214. The machine-readable tag 200 may transmit data from the chip 204 via the antenna coil 202, which is captured by the antenna coil 102 and read by the circuitry 104 of the wearable device 100. In some embodiments, the data may optionally be transferred from the circuitry 104 to a remote computing device 702 for processing or storage as shown in FIG. 23. Otherwise, the data may be transferred from the circuitry 104 to a processing device 148 of the wearable device for processing or storage as shown in FIG. 27.

The resonant members 212 may be vibratable by a Lorentz force. The Lorentz force is the force that acts on a charged particle travelling through an orthogonal magnetic field. In this instance, a magnetic field is applied to the resonant members 212 in a direction perpendicular to the current flow through the electrical conductor 214. In some embodiments, the machine-readable tag 200 may further include a magnet 206 or element by which a magnetic field is applied orthogonally to the machine-readable tag 200, as shown in FIG. 23. For example, the magnet 206 may be positioned adjacent and beneath the chip 204 in the machine-readable tag 200. Alternatively, the magnet 206 may be included in the container 600 associated with the machine-readable tag 200 (not shown).

FIG. 24 depicts a vibratable member 218 in the form of a bridge structure 222 including a beam 228 supported by two columns 224 and 226 projecting from a substrate 220. The electrically conductive path 214 as shown forms part of the electrical circuit of the chip 204 as shown in FIG. 23. Since the current in the conductor 214 is an alternating current, the orthogonal force generated due to the magnetic field (such as by magnet 206) is also an alternating force, resulting in the vibration of the beam 228. If the frequency of the alternating current in the conductor 214 is at or near the resonant frequency of the beam 228, the beam 228 will vibrate. The vibratable members 218 are described in more detail in International Patent Application No. WO 2004/084131, to the present Applicant, the entire contents of which are incorporated herein by reference.

Referring now to FIG. 25, each of the resonant members 212 forming part of the machine-readable tag 200 have a notional resonant frequency corresponding to one of a predetermined number of resonant frequencies f₁, f₂, f₃, etc. Preferably, the resonant frequencies f₁, f₂, f₃, etc. are in a different frequency range. If the circuitry 104 detects a resonant frequency at any of the frequency positions f₁ onwards, the circuitry 104 interprets that resonant frequency as a binary “1”. By contrast, the absence of a resonant frequency at any of those predetermined frequency positions is interpreted as a binary “0”. The sequence of binary 1's and 0's detected by the circuitry 104 corresponds to the machine-readable identifier 230. The reading of machine-readable tags 200 having resonant members 212, 218 are described in greater detail in International Patent Application No. WO 2010/037166, to the present Applicant, the entire contents of which are incorporated herein by reference.

The machine-readable tag 200 having the machine-readable identifier 230 may be replaced with an active or passive RFID tag which does not necessarily include a MEMS structure, such as a CMOS based RFID tag. For example, a resistor having a temperature-dependant value could form part of the tag and that value be read. Alternatively, an antenna forming part of the machine-readable tag 200 may have a temperature-dependant impedance which is detectable by a tuned antenna. A person skilled in the art will be able to conceive of a variety of machine-readable tags 200 which are suitable for use in the context of the present invention.

Additional functions of the wearable device 100 will now be described with reference to FIGS. 27 and 28, which illustrate schematic diagrams of the elements and componentry of the wearable device 100 according to embodiments of the invention

In some embodiments, the wearable device 100 may further include a user activation device for triggering the circuitry 104 to activate the antenna 102 and wirelessly read data from the machine-readable tag 200. The user activation device may include a pressure switch 172 as shown in FIG. 28, which may be manually operable by the user 400. Preferably, the pressure switch 172 is positioned near the antenna 102, and ideally, in the reader head 118 of a fingertip antenna housing portion 108, as shown in FIG. 2. The pressure switch 172 may include a depressible button for manually applying pressure thereto when the user 400 desires to trigger the circuitry 104 to read the machine-readable tag 200.

In some embodiments, the circuitry 104 may be adapted to automatically activate the antenna 102 and wirelessly read data from the machine-readable tag 200. The circuitry 104 may be adapted to automatically activate the antenna 102 by detection of a magnetic field associated with the machine-readable tag 200. For example, the machine-readable tag 200 may include a magnet 206 as shown in FIG. 23. The circuitry 104 may detect a strength of the magnetic field associated with the magnet 206 and automatically activate the antenna 102 when the strength exceeds a threshold value or range of values. This may be particularly useful in circumstances where the machine-readable tag 200 is in a difficult or unsafe area for the user 400 to access, such as inside a tank 602 filed with liquid nitrogen.

In other embodiments, the circuitry 104 may automatically activate the antenna 102 at regular intervals, for example every second, to wirelessly read data from machine-readable tags 200 near the wearable device 100. The circuitry 104 may also direct varying levels of current through the antenna 102 in order to detect a spectrum of frequencies of the machine-readable tag 200. Automatic activation of the antenna 102 may allow the user 400 having the wearable device 100 to move through the temperature-controlled environment and automatically read machine-readable tags 200 at a distance therefrom.

As shown in FIG. 28, the circuitry 104 may be in communication with multiple antenna coils, such as antenna coil 102 of the primary reader 118, antenna coil 140 of the secondary or wand reader 170 and an antenna coil 184 of the vial reader 154. The circuitry 104 may be adapted to activate one or more of the antenna coils to read machine-readable tags 200. Although only one circuitry 104 is shown in FIG. 28, it would be readily appreciated by a person skilled in the art that each reader 118, 170 and 154 may include circuitry components 104 for individually activating the antennas 102, 140 and 184, respectively. The componentry shown in FIG. 28 may be illustrative of componentry of the wearable device 100 as shown in FIG. 22.

As previously mentioned, the wearable device 100 may include a processing device 148, which may be in communication with the circuitry 104 as shown in FIGS. 27 and 28. The processing device 148 may be any known Computer Processing Unit (CPU) as would be appreciated by a person skilled in the art. For example, the processing device 148 may include a Celeron chip by Intel Corporation located, for example, on an ETX form factor PCB, or a controller, such as a microcontroller. The processing device 148 may be adapted to be in communication with a storage device 168, which may include a memory device having a non-volatile memory, such as a hard drive. The processing device 148 may be adapted to execute instructions stored in the storage device 168 for processing data at the wearable device 100.

In some embodiments, the wearable device 100 may optionally include a communications device 150 in communication with one or both of the circuitry 104 and the processing device 148. The communications device 150 may be adapted to transmit the data wirelessly read by the circuitry 104 directly to a remote computing device 702 without or without processing by the processing device 148. Feedback may be provided by the remote computing device 702, and optionally, transmitted back to the wearable device 100 via the communications device 150. Alternatively, the communications device 150 may be adapted to receive feedback on the data wirelessly read from the processing device 148 and transmit the feedback to the remote computing device 702. The communications device 150 may also be adapted to transmit additional information, such as location information, temperature measurements and identification of the wearable device 100 to the remote computing device 702. The communications device 150 may include an antenna suitable for transmitting data from the wearable device 100 to the remote computing device 702 via a network device 704 (see also FIG. 30).

The processing device 148 may provide feedback on the data wirelessly read to the user 400 by means of an output device 152 as shown in FIG. 28 and previously described. Notably, the display device 122 may include an input/output device 178 for receiving input from the user 400. The display device 122 may receive, for example, login details from the user 400 including a username and password. Additionally/alternatively, the display device 122 may receive user instructions, such as to transmit data and/or feedback to the remote computing device 702 or store data and/or feedback locally in the storage device 168.

The wearable device 100 may also include an on/off switch 126 operable by the user 400 to switch a power source 166 of the wearable device 100 on or off as shown in FIG. 28. The power source 166 is preferably a battery for wireless use of the wearable device 100 in the temperature-controlled environment. Alternatively, the power source 166 may be a hard-wired connection to an electricity outlet, for example.

The processing device 148 may be adapted to receive temperature measurements from one or more temperature sensors 162 of the wearable device. The temperature sensors 162 may be located in one or both of the vial reader 154 and reader head 118 housing the antenna 102, or elsewhere in the wearable device 100. The processing device 148 may be adapted to monitor viability of a temperature-sensitive item stored in a container 600 associated with the machine-readable tag 200. This may include determining if the temperature measurements exceed a required storage temperature, such as a threshold or range of values, for the temperature-sensitive item. The processing device 148 may provide feedback to the user 400 on the viability of the temperature-sensitive item, such as by outputting through the output device 152 temperature measurements and/or an alarm if the required storage temperature has been exceeded.

The processing device 148 may also be adapted to receive humidity measurements from one or more humidity sensors 190 and/or gas concentration measurements from one or more gas sensors 192 of the wearable device 100. The processing device 148 may be further adapted to monitor moisture content and/or gas concentration near a container 600 associated with the machine-readable tag 200. For example, this may include determining if the humidity measurements and/or gas concentration measurements exceed a required storage humidity and/or storage gas concentration, which may include a threshold or range of values. The processing device 148 may be further adapted to provide feedback to the user 400 on the potential for moisture build-up near the container 600 and/or whether the gas concentration in the surrounding environment is safe for the user 400. For example, the processing device 148 may output through the output device 152 humidity and/or gas concentration measurements and/or an alarm if the required storage humidity and/or gas concentration has been exceeded.

The processing device 148 may also be adapted to receive location information of the machine-readable tag 200 from the remote computing device 702 and to output the location information to the user 400 for locating the machine-readable tag 200 in the temperature-controlled environment. The processing device 148 may output the location information to the user 400 through the output device 152, such as by displaying the location of the machine-readable tag 200 on a display device 122.

The wearable device 100 may also include a location device 174 for providing location information of the wearable device 100 as shown in FIG. 28. The location device 174 may include a Global Positioning System (GPS) sensor. The processing device 148 may be further adapted to receive the location information of the wearable device 100 and to output directions to the user 400 for locating the machine-readable tag 200 in the temperature-controlled environment. The processing device 148 may cause the display 124 on the display device 122 to display the location of the wearable device 100 on a map and highlight directions to the machine-readable tag 200. Advantageously, these features may assist the user 400 in locating the machine-readable tag 200 in temperature-controlled environments that are dark or poorly lit or numerous containers 600 associated with machine-readable tags 200. Thus, the location information may enable the user 400 to more quickly and efficient perform the task of wirelessly reading the machine-readable tags 200.

Furthermore, in some embodiments the wearable device 100 further includes an identification device 176, which is readable by a remote computing device 702. The remote computing device 702 may use the identification to verify the identification of the wearable device 100 using a database and authorise user access to the temperature-controlled. The identification device 176 may include a machine-readable tag, such as an RFID tag, which is adapted to be wirelessly read by the remote computing device 702. The identification device 176 may include identification information such as a device number, year and date of safety inspection.

The wearable device 100 may also include one or more internal temperature sensors 188 for measuring a temperature of the upper limb 410 of the user 400. The internal temperature sensors 188 may be housed in the glove 500, such as inside the glove housing 512 for contact with the user's arm 410. The internal temperature sensors 188 may be located on an inner surface 516 of the glove 500 and/or within the removable sleeve 518, as shown in FIG. 8. Preferably, the internal temperature sensors 188 are positioned near the reader head 118 housing the antenna 102. For example, the internal temperature sensors 188 may be located in the glove housing 512 at the fingertips 504 in embodiments of wearable device 100 having a fingertip antenna 102. The processing device 148 may receive temperature measurements from the internal temperature sensors 188. The processing device 148 may be adapted to monitor the temperature measurements for determining exposure of the user's upper limb 410 to extreme temperatures in the temperature-controlled environment. The processing device 148 may be adapted to determine if the temperature measurements exceed a threshold or range of values indicating an unsafe temperature and provide feedback to the user 400, such as by using the output device 152, of the temperature measurements and/or an alarm for safe use.

In some embodiments, the processing device 148 may be adapted to monitor the temperature measurements to determine if the wearable device 100 is being worn on a user's upper limb 410. The processing device 148 may be adapted to determine if the temperature measurements exceed a threshold or range of values indicating that the user 400 is wearing the wearable device 100. In other embodiments (not shown), the wearable device 100 may include internal sensors for measuring contact of the user's upper limb 410 with the wearable device 100, such as force sensors or electrical resistance sensors. The processing device 100 may be adapted to use the data from the internal sensors for controlling the power source 166. When the processing device 100 determines that the user 400 is wearing the wearable device 100, the power source 166 may be activated by the processing device 100 from a sleep mode to an active mode. Otherwise, the processing device 100 may direct the power source 166 to remain in a sleep mode to conserve power.

FIG. 29 illustrates a system 700 for wirelessly reading data in a temperature-controlled environment according to a preferred embodiment of the invention. The system includes a wearable device 100 for wirelessly reading data in a temperature-controlled environment. The wearable device 100 is adapted to be worn on an upper limb 410 of a user 400. The wearable device 100 may include an antenna 102 and circuitry 104 adapted to activate the antenna 102 and wirelessly read data from a machine-readable tag 200 in the temperature-controlled environment. The system 700 also includes a remote computing device 702 in communication with the wearable device 100. The remote computing device 702 is adapted to receive data wirelessly read from the machine-readable tag 200 in the temperature-controlled environment.

The wearable device 100 may include one or more of the features described in the previous embodiments, including various combinations thereof. Furthermore, the remote computing device 702 may perform similar functions to the processing device 148 as described with regard to the embodiments of FIGS. 27 and 28.

Preferably, the remote computing device 702 is in communication with the communication device 150 of the wearable device 100, as shown in FIG. 27, for receiving data from the wearable device 100. The data received may include the data wirelessly read from the machine-readable tag 200 and/or feedback thereof, temperature measurements, humidity measurements, gas concentration measurements, location information and identification information. The data can be downloaded or otherwise electronically transmitted to the remote computing device 702 either live or at some subsequent time. For example, the remote computing device 100 may be further adapted to wirelessly communicate with the wearable device 100 through a network device 704 as shown in FIG. 30.

The remote computing device 702 may be further adapted to update inventory records using the data received from the wearable device 100. Accordingly, a permanent data log can be maintained throughout various storage, processing and transport activities that the machine-readable tag 200 may be subject to over its life. In some embodiments, the data recordings may be made continuously and the data transmitted to the remote computing device 702, such as a server 710 or workstation 720, as shown in FIG. 30, periodically or streamed continuously as required. Notably, the workstation 720 may include a computing device in the temperature-controlled environment, which can be accessed by the user 400.

In some embodiments, the remote computing device 702 may be adapted to receive temperature measurements from one or more temperature sensors 162 of the wearable device 100, as shown in FIG. 28. The temperature measurements may be from temperature sensors 162 in the vial reader 154 or elsewhere in the wearable device 100. The remote computing device 702 may use the temperature measurements to monitor viability of a temperature-sensitive item stored in the container 600 or vial 612 and transmit feedback on the viability of the temperature-sensitive item to the wearable device 100.

The remote computing device 702 may also be adapted to receive temperature measurements from one or more internal temperature sensors 188 of the wearable device 100, as shown in FIG. 28. The remote computing device 702 may use the temperature measurements to monitor exposure of the user's upper limb 410 to extreme temperatures in the temperature-controlled environment and to transmit feedback on the exposure to the wearable device 100. Furthermore, the remote computing device 702 may use the temperature measurements to detect if the user 400 is wearing the wearable device 100, such as for compliance with PPE requirements. Additionally/alternatively, the remote computing device 702 may use contact measurements from force sensors or electrical resistance sensors of the wearable device 100 to determine if the user 400 is wearing the wearable device 100.

The remote computing device 702 may also be adapted to receive humidity measurements from one or more humidity sensors 190 and/or gas concentration measurements from one or more gas sensors 192 of the wearable device 100. The remote computing device 702 may use the humidity and/or gas concentration measurements to monitor moisture content and/or gas concentration near a container 600 associated with the machine-readable tag 200 and to transmit feedback on the potential for moisture build-up near the container 600 and/or whether the gas concentration in the surrounding environment is safe for the user 400 to the wearable device 100.

In some embodiments, the remote computing device 702 may be further adapted to transmit location information of the machine-readable tag 200 to the wearable device 100 for locating the machine-readable tag 200 in the temperature-controlled environment. The remote computing device 702 may be further adapted to receive the location information of the wearable device 100 and to track the location of the wearable device 100 in the temperature-controlled environment.

In some embodiments, the remote computing device 702 may be further adapted to read the identification of the wearable device 100 from the identification device 176. The remote computing device 702 may be further adapted to verify the identification of the wearable device 100 using a database and authorise user access to the temperature-controlled environment. Advantageously, this may allow the wearable device 100 to be used in an identification system for preventing users without PPE, e.g., without the wearable device 100, from entering restricted areas in a laboratory, for example. The identification system may be used to demonstrate compliance with PPE requirements. The remote computing device 702 may store the data and provide a data log for satisfying safety and operating procedures. The data log may include a record of user entry and exit from the temperature-controlled environment, together with an indication that the user 400 was wearing the wearable device 100, and data records from machine-readable tags 200 read while the user 400 was in the temperature-controlled environment. Accordingly, the system 700 can be used to ensure that only trained users 400, which are recorded in the database, are allowed to enter the temperature-controlled environment and use the wearable device 100.

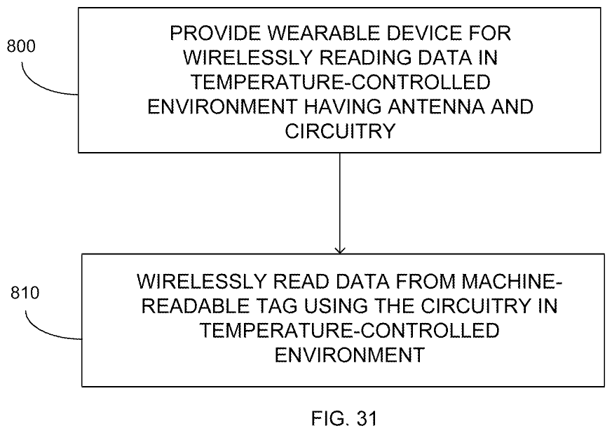

FIG. 31 illustrates a method for wirelessly reading data in a temperature-controlled environment according to a preferred embodiment of the invention. The method includes the step 800 of providing a wearable device 100 for wirelessly reading data in a temperature-controlled environment. The wearable device 100 is adapted to be worn on an upper limb 410 of a user 400. The wearable device 100 includes an antenna 102 and circuitry 104 adapted to activate the antenna 102 and wirelessly read data from a machine-readable tag 200 in the temperature-controlled environment. The method also includes the step 810 of wirelessly reading data from the machine-readable tag 200 using the circuitry 104 in the temperature-controlled environment. The wearable device 100 may include one or more of the features described in the previous embodiments, including various combinations thereof.

In some embodiments, the step 810 of wirelessly reading data may include detecting changes in resonance of the machine-readable tag 200 using the circuitry 104. The method may further include applying an excitation signal to the machine-readable tag 200 through the antenna 102 using the circuitry 104 that vibrates the resonant members 212 and wirelessly reading the identifier 230 using the circuitry 104, as shown in FIG. 24. The method may further include detecting a magnetic field associated with the machine-readable tag 200 using the circuitry 104 and automatically activating the antenna 102 based on the detected magnetic field.

Additionally/alternatively, the method may include a user 400 operating a user activation device of the wearable device 100 to trigger the circuitry 104 and activate the antenna 102 and wirelessly read data from the machine-readable tag 200. This may include the user 400 manually operating a pressure switch 172 to trigger the circuitry 104, such as by depressing a button on the pressure switch 172. In some embodiments, the method may alternatively include automatically activating the antenna 102 at regular intervals, using the processing device 148, to wirelessly read data from the machine-readable tag 200.

In some embodiments, the method may further include providing directed illumination towards the machine-readable tag 200 or the wearable device 100 for visibility in the temperature-controlled environment. This may include the user 400 activating the tag illuminator 132 or device illuminator 134, or both illuminators 180, for providing the directed illumination.

The method may further include extending or removing the antenna 102 from the wearable device 100 and positioning the antenna 102 near the machine-readable tag 200 in the temperature-controlled environment.

Additionally/alternatively, the method may further include extending or removing the second antenna 140 housing in the secondary or wand reader 170 from the wearable device 100 and positioning the second antenna 140 near the machine-readable tag 200 in the temperature-controlled environment. The user 400 may extend or remove the antenna 102 and/or second antenna 140 from the wearable device 100. The method may also include the user 400 positioning a container 600 associated with the machine-readable tag 200 near the antenna 102 using one or more tools 136 from the docking station 138.

In some embodiments, the method further includes receiving a vial 612 associated with the machine-readable tag 200 in the vial reader 154 and wirelessly reading data from the machine-readable tag 200 using the circuitry 104 in the temperature-controlled environment. The method may further include measuring the temperature of the container 600 or optionally the vial 612 using the one or more temperature sensors 162. The method may further include using the processing device 148 for one or more of: receiving temperature measurements from the one or more temperature sensors 162; monitoring viability of a temperature-sensitive item stored in the container 600; and providing feedback to the user 400 on the viability of the temperature-sensitive item. The method may also include using the remote computing device 702 in communication with the wearable device 100 for one or more of: receiving temperature measurements from the one or more temperature sensors 162; monitoring viability of the temperature-sensitive item stored in the container 600; and transmitting feedback on the viability of the temperature-sensitive item to the wearable device 100 for the user 400.

In some embodiments, the method may further include measuring humidity and/or gas concentration of the surrounding environment near the container 600 using the one or more humidity sensors 190 and/or gas sensors 192. The method may also include using the processing device 148 for one or more of: receiving humidity and/or gas concentration measurements from the one or more humidity sensors 190 and/or gas sensors 192; monitoring moisture content and/or gas concentration near the container 600; and providing feedback to the user 400 on the potential for moisture build-up near the container 600 and/or whether the gas concentration in the surrounding environment is safe for the user 400. The method may also include using the remote computing device 702 in communication with the wearable device 100 for one or more of: receiving humidity measurements from one or more humidity sensors 190 and/or gas concentration measurements from one or more gas sensors 192 of the wearable device 100; monitoring moisture content and/or gas concentration near a container 600 associated with the machine-readable tag 200; and transmitting feedback on the potential for moisture build-up near the container 600 and/or whether the gas concentration in the surrounding environment is safe for the user 400 to the wearable device 100.

Furthermore, the method may include using the processing device 148 for: receiving temperature measurements from one or more internal temperature sensors 188 of the wearable device 100; monitoring exposure of the user's upper limb 410 to extreme temperatures in the temperature-controlled environment; and providing feedback on the exposure to the user 400. The method may also include using the remote computing device 702 for: receiving temperature measurements from one or more internal temperature sensors 188 of the wearable device 100; monitoring exposure of the user's upper limb 410 to extreme temperatures in the temperature-controlled environment; and transmitting feedback on the exposure to the wearable device 100. The method may further include using the processing device 148 or remote computing device 702 to determine if the user 400 is wearing the wearable device 100 based on the temperature measurements and/or contact measurements from force sensors or electrical resistance sensors of the wearable device 100.

The method may also include using the processing device 148 for providing feedback on the data wirelessly read by the circuitry 104. The feedback may include one or both of whether the data received is sufficient for reading of the machine-readable tag 200 and an identifier 230 of the machine-readable tag 200. The method may also include using the processing device 148 to transmit the feedback to the remote computing device 702 using the communications device 150. The processing device 148 may provide one or more of visual, auditory or sensory feedback to the user 400 using the output device 152 of the wearable device 100. The method may also include wirelessly reading data representative of one or both of indicia and alphanumeric data using an optical device 158 of the wearable device 100, as shown in FIG. 28.

In some embodiments, the method may also include using the processing device 148 for: receiving location information of the machine-readable tag 200 from the remote computing device 702; and outputting the location information to the user 400 for locating the machine-readable tag 200 in the temperature-controlled environment. The method may also include using the processing device 148 to receiving location information of the wearable device 100 from the location device 176; and outputting directions to the user 400 for locating the machine-readable tag 200 in the temperature-controlled environment.

In some embodiments, the method may also include using the remote computing device 702 for: receiving location information of the wearable device 100 from the location device 176; and tracking the location of the wearable device 100 in the temperature-controlled environment. The method may further include using the remote computing device 702 for: reading the identification of the wearable device 100; verifying the identification of the wearable device 100 using a database; and authorising user access to the temperature-controlled environment based on the verification.

The inventive device, system and method may advantageously enable wireless reading of machine-readable tags, such as RFID tags, in temperature-controlled environments, particularly low-temperature and cryogenic storage facilities. The inventive wearable device includes a housing suitably shaped for use on the user's upper limb that desirably enables one-handed operation, allowing a free-hand for use in other actions. The wearable device may be able to withstand extreme temperatures by the construction of the housing which provides protection from one or both of thermal shock and fluid damage. The circuitry of the wearable device being adapted to wirelessly read data by detecting changes in resonance of the machine-readable tag enables use in low-temperature or cryogenic environments. Furthermore, the wearable device is adapted to facilitate access to machine-readable tags in the temperature-controlled environment by providing illumination and facilitating access of the machine-readable tag near the antenna reader and vice versa through inventive features of the wearable device. Finally, the wearable device enables temperature monitoring of containers associated with the machine-readable tag and of the wearable device and the user, together with location and identification detection for providing safe and controlled use of the wearable device in the temperature-controlled environment.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components.

It is to be understood that various modifications, additions and/or alternatives may be made to the parts previously described without departing from the ambit of the present invention as defined in the claims appended hereto.

It is also to be understood that the following (provisional) claims are provided by way of example only, and are not intended to limit the scope of what may be claimed in any such future application. Features may be added to or omitted from the (provisional) claims at a later date so as to further define or re-define the invention or inventions.

1-47. (canceled)

48. A wearable device for wirelessly reading data in a temperature-controlled environment, the wearable device being adapted to be wom on an upper limb of a user, wherein the wearable device includes an antenna and circuitry adapted to activate the antenna and wirelessly read data from a machine-readable tag in the temperature-controlled environment.

49. The wearable device according to claim 48, further including a housing for the antenna and the circuitry, wherein the housing is shaped to be worn on the upper limb of the user.

50. The wearable device according to claim 49, wherein the housing includes one or both of:
an antenna housing portion for housing the antenna, wherein the antenna housing portion is shaped to be worn on a hand of the user; and
a circuitry housing portion for housing the circuitry, wherein the circuitry housing portion is shaped to be worn on a hand, a wrist or an arm of the user.

51. The wearable device according to claim 48, wherein one of:
the wearable device is adapted to be retrofit to a glove; or the wearable device is a glove.

52. The wearable device according to claim 48, wherein the housing includes one or more of:
a protective layer for isolating one or both of the antenna and the circuitry from the temperature-controlled environment to minimise one or both of thermal shock and fluid damage in the temperature-controlled environment;
one or more conduits for directing fluid away from one or both of the antenna and the circuitry for drainage externally of the wearable device to minimise one or both of thermal shock and fluid damage in the temperature-controlled environment; and
a hydrophobic coating to deter fluid collection on a surface thereof for visibility of the wearable device in the temperature-controlled environment.

53. The wearable device according to claim 48, wherein the circuitry is adapted to wirelessly read the data by detecting changes in resonance of the machine-readable tag, wherein the machine-readable tag includes a plurality of resonant members encoding an identifier, and wherein the circuitry is further adapted to one or both of:
apply an excitation signal to the machine-readable tag through the antenna that vibrates the resonant members to wirelessly read the identifier, and
automatically activate the antenna by detection of a magnetic field associated with the machine-readable tag.

54. The wearable device according to claim 48, further including one or more illuminators for providing directed illumination towards the machine-readable tag or the wearable device for visibility in the temperature-controlled environment, wherein the one or more illuminators are positioned near the antenna.

55. The wearable device according to claim 48, wherein one or both of:
the antenna is extendable or removable from the wearable device for positioning near the machine-readable tag in the temperature-controlled environment; and
the wearable device further includes a second antenna that is extendable or removable from the wearable device for positioning near the machine-readable tag in the temperature-controlled environment.

56. The wearable device according to claim 48, further including a docking station having one or more tools operable by the user for positioning a container associated with the machine-readable tag near the antenna.

57. The wearable device according to claim 48, further including a vial reader for receiving a vial associated with the machine-readable tag, wherein the vial reader includes an antenna operable to be activated by circuitry for wirelessly reading data from the machine-readable tag.

58. The wearable device according to claim 48, further including one or more temperature sensors for measuring a temperature of a container associated with the machine-readable tag, and wherein the wearable device further includes a processing device adapted to one or more of:
receive temperature measurements from the one or more temperature sensors;
monitor viability of a temperature-sensitive item stored in the container; and
provide feedback to the user on the viability of the temperature-sensitive item.

59. The wearable device according to claim 48, further including a processing device in communication with a remote computing device, wherein the processing device is adapted to receive location information of the machine-readable tag from the remote computing device and to output the location information to the user for locating the machine-readable tag in the temperature-controlled environment.

60. The wearable device according to claim 59, further including a location device for providing location information of the wearable device, wherein the processing device is further adapted to receive the location information of the wearable device and to output directions to the user for locating the machine-readable tag in the temperature-controlled environment.

61. The wearable device according to claim 48, further including an identification device for providing an identification of the wearable device, wherein the identification is readable by a remote computing device for verifying the identification of the wearable device using a database and authorising user access to the temperature-controlled environment based on the verification.

62. A system for wirelessly reading data in a temperature-controlled environment, the system including:
a wearable device for wirelessly reading data in a temperature-controlled environment, the wearable device being adapted to be worn on an upper limb of a user, wherein the wearable device includes an antenna and circuitry adapted to activate the antenna and wirelessly read data from a machine-readable tag in the temperature-controlled environment; and
a remote computing device in communication with the wearable device, wherein the remote computing device is adapted to receive data wirelessly read from the machine-readable tag in the temperature-controlled environment.

63. The system according to claim 62, wherein the wearable device further includes one or more temperature sensors for measuring a temperature of a container associated with the machine-readable tag, and wherein the remote computing device is further adapted to one or more of:
receive temperature measurements from the one or more temperature sensors;
monitor viability of a temperature-sensitive item stored in the container; and
transmit feedback on the viability of the temperature-sensitive item to the wearable device.

64. The system according to claim 62, wherein the remote computing device is further adapted to transmit location information of the machine-readable tag to the wearable device for locating the machine-readable tag in the temperature-controlled environment.

65. The system according to claim 64, wherein the wearable device further includes a location device for providing location information of the wearable device, and wherein the remote computing device is further adapted to receive the location information of the wearable device and to track the location of the wearable device in the temperature-controlled environment.

66. The system according to claim 62, wherein the wearable device further includes an identification device for providing an identification of the wearable device, and wherein the remote computing device is further adapted to read the identification of the wearable device, verify the identification of the wearable device using a database and authorise user access to the temperature-controlled environment.

67. A method for wirelessly reading data in a temperature-controlled environment, the method including the steps of:
providing a wearable device for wirelessly reading data in a temperature-controlled environment, the wearable device being adapted to be worn on an upper limb of a user, wherein the wearable device includes an antenna and circuitry adapted to activate the antenna and wirelessly read data from a machine-readable tag in the temperature-controlled environment; and
wirelessly reading data from the machine-readable tag using the circuitry in the temperature-controlled environment.
